(19) United States
(12) Patent Application Publication
(10) Pub. No.: US 2021/0353907 A1
(43) Pub. Date: Nov. 18, 2021

(54) CONSTRUCTIONS FOR A DEFLECTABLE SHAFT CATHETER

(71) Applicant: Medtronic, Inc., Minneapolis, MN (US)

(72) Inventors: Zhongping Yang, Woodbury, MN (US); Ronald A. Drake, St. Louis Park, MN (US); Robert Kowal, Excelsior, MN (US); Megan L. Platner, Eden Prairie, MN (US); Lester O. Stener, Hudson, WI (US)

(21) Appl. No.: 17/319,435

(22) Filed: May 13, 2021

Related U.S. Application Data
(60) Provisional application No. 63/025,256, filed on May 15, 2020.

(51) Int. Cl. A61M 25/01; A61M 25/00

(52) U.S. Cl. CPC A61M 25/0136; A61M 2025/0188; A61M 25/0097

(57) ABSTRACT

A deflectable shaft catheter may include a handle extending laterally from a hub of the catheter to form an acute angle therewith, wherein the handle contains a wire controller and a guide that rotates in plane with the angle. A spring biased slider of the wire controller, for the aforementioned handle, or an in-line handle sans the guide, may include first and second parts between which a proximal end of the wire extends and bends into a bore formed in the first part. Alternately, the slider includes an elastomeric core sandwiched between first and second parts thereof. The slider may further include a cavity in which a spring member biases the proximal end of the wire toward a distal end of the cavity, but only forces the wire to the cavity distal end, if a predetermined spring force of the member is greater than an opposing force along the wire.

### RELATED APPLICATIONS

The present application claims the benefit of U.S. Provisional Application No. 63/025,256, filed May 15, 2020, which is incorporated herein by reference in its entirety.

### FIELD OF THE DISCLOSURE

The present disclosure pertains to interventional medical systems, and more particularly to constructions of deflectable shaft catheters.

### BACKGROUND

Those skilled in the art of interventional medicine are familiar with various types of deflectable shaft catheters used to deliver medical therapy and/or provide medical monitoring. In many cases, a shaft of such a catheter has a lumen in which an elongate wire extends. A distal end of the wire is coupled to the shaft in proximity to a distal end thereof, and a proximal end of the wire is coupled to a wire controller. The wire controller may be mounted in a handle of the catheter so that an operator who grasps the handle may actuate the wire controller to deflect the catheter shaft via movement of the wire within the wire lumen. The shaft deflection can help the operator to maneuver the distal end of the catheter shaft toward a target site within a body of a patient. Although a variety of constructions suitable for deflectable shaft catheters are known in the art, there is still a need for new and improved constructions, for example, to increase efficiency in manufacturing while enhancing the handling of these catheters.

### SUMMARY

According to some embodiments of a first type disclosed herein, a handle of a deflectable shaft catheter, which contains a wire controller coupled to a proximal end of an elongate wire of the catheter, extends laterally from a hub coupled to a proximal end of the deflectable shaft. In these embodiments, a first axis, which is defined by a delivery lumen of the catheter, crosses a second axis. The second axis is defined by the lateral extent of the handle, to form an acute angle between the handle and the hub and a rotating guide, which is mounted within the handle and has an external groove in which the wire extends between the shaft and the wire controller, rotates in plane with the first and second axes. The hub of the deflectable catheter of the first type of embodiments may include a relatively thin wall section located opposite from the handle, for example, to facilitate slitting of the hub and catheter shaft. Furthermore, the hub may have a tapered profile at a proximal end thereof, for example, to orient a proximal opening of a delivery lumen of the catheter toward a thumb of a hand whose fingers grasp around the handle. However, according to some alternate embodiments of a second type disclosed herein, a handle of a deflectable shaft catheter extends in-line with the deflectable shaft thereof and may not include the hub features or the rotating guide. Various embodiments of wire controllers for deflection assemblies disclosed herein may be incorporated by either type of embodiment.

A deflection assembly, according to the disclosed embodiments of deflectable shaft catheters, includes an elongate wire and a wire controller coupled thereto. The wire controller, according to preferred embodiments, is formed by a spring biased slider that is mounted within a shell forming the handle of the catheter. The handle shell extends over a length from a proximal end thereof, which is coupled to the catheter shaft, to a distal end thereof and includes a slot extending therethrough, from an inner surface to an outer surface of the shell. A length of the slot extends lengthwise along the handle. The handle shell further includes an engagement feature formed in the inner surface thereof, in proximity to the slot, and a support surface formed in the inner surface thereof, opposite the slot. The spring biased slider is configured for mounting in between the slot and the support surface of the handle shell so that a first side of the slider faces the support surface, a second side of the slider faces the engagement feature, and an operator interface, which extends from the second side of the slider, extends through the slot to protrude from the outer surface of the shell. When the slider is relaxed according to the spring bias thereof, the second side of the slider interlocks with the engagement feature of the handle shell to prevent movement of the slider along the length of the slot; and, when the slider is deformed against the spring bias thereof, responsive to a force being applied to the operator interface, the second side of the slider moves away from the engagement feature of the handle and no longer interlocks therewith, and the first side of the slider slides along the support surface as the slider moves along the length of the slot. According to some embodiments, the spring biased slider includes first and second parts snap-fit together. A proximal end of the elongate wire extends between the first and second parts and bends into a bore formed in the first part for the coupling of the wire to the wire controller. In these embodiments, the first part may include opposing cantilever beam members that define the spring bias of the slider. According to some alternate embodiments, the spring biased slider includes an elastomeric core that is sandwiched between first and second parts thereof to define the spring bias of the slider. In these alternate embodiments, the proximal end of the elongate wire may extend along first side of the slider, formed by the first part, and bend into a bore formed in the first part for the coupling of the wire to the wire controller.

According to some additional embodiments, the wire controller includes a spring-biased slider that has a cavity (in addition to the aforementioned first and second sides and operator interface) in which the proximal end of the wire is coupled and a spring member of the controller is mounted. When the slider of these embodiments is mounted in the handle shell, as described above, the cavity is located between the support surface and the engagement feature of the handle shell and has a proximal end oriented toward the proximal end of the handle shell and a distal end oriented toward the distal end of the handle shell. The spring member of these embodiments is biased to push the proximal end of the wire toward the distal end of the cavity. The bias is governed by a predetermined spring force of the spring member. The interlocking with the engagement feature of the handle shell and the movement in response to the applied force, as described above, are the same for the slider of these embodiments, but, when a component of a vector of the applied force is in the distal direction, the slider pushes the wire in a distal direction only if the predetermined spring force of the spring member is large enough to overcome an opposing force applied along a length of the wire.

### BRIEF DESCRIPTION OF THE DRAWINGS

The following drawings are illustrative of particular embodiments of the present disclosure and therefore do not limit the scope of the invention. The drawings are not to scale (unless so stated) and are intended for use in conjunction with the explanations in the following detailed description. Embodiments will hereinafter be described in conjunction with the appended drawings wherein like numerals denote like elements, and:

FIG. 1A is a plan view of deflectable shaft catheter, according to some embodiments of the present invention;

FIG. 1B is a schematic depicting the catheter of FIG. 1A positioned within a patient's venous system;

FIG. 2A is a schematic showing a hand of an operator handling the catheter of FIG. 1A, according to some embodiments;

FIG. 2B is a schematic showing an injection syringe joined to the catheter of FIG. 1A, according to some embodiments;

FIG. 2C is a schematic showing a slitting tool positioned for removing the catheter of FIG. 1A from around a medical electrical lead, according to some embodiments;

FIG. 3A is a longitudinal cross-section view with an enlarged detail view of a portion of the catheter of FIG. 1A, according to some embodiments;

FIG. 3B is an exploded perspective view of a handle assembly for the catheter of FIG. 1A, according to some embodiments;

FIG. 3C is a perspective view of a first part of a wire controller isolated from the exploded view of FIG. 3B, according to some embodiments;

FIG. 3D is a perspective view of a second part of the wire controller isolated from the exploded view of FIG. 3B, according to some embodiments;

FIG. 3E is a perspective view of an elongate wire and a rotating guide isolated from the exploded view of FIG. 3B, according to some embodiments;

FIG. 3F is a perspective view of a first part of a handle shell isolated from the exploded view of FIG. 3B;

FIG. 3G is a perspective view of a second part of a handle shell isolated from the exploded view of FIG. 3B;

FIG. 4A is a perspective view of a deflectable shaft catheter, according to some alternate embodiments of the present invention;

FIG. 4B is a longitudinal cross-section view with an enlarged detail view of a portion of the catheter of FIG. 4A, according to some embodiments;

FIG. 4C is an exploded perspective view of a handle shell of the catheter of FIG. 4A, according to some embodiments;

FIG. 4D is a longitudinal cross-section view of an isolated portion of the catheter of FIG. 4A;

FIG. 4E is an exploded perspective view of a wire controller that may be employed by the catheter of FIG. 4A, according to some embodiments;

FIG. 4F is a plan view of the wire controller of FIG. 4E, according to some embodiments;

FIG. 4G is a perspective view of a component that may be employed by the wire controller of FIG. 4E, according to some embodiments;

FIG. 5A is a plan view of a portion of a deflectable shaft catheter, according to some additional embodiments of the present invention;

FIG. 5B is a cross-section view through section line B-B of FIG. 5A, according to some embodiments;

FIG. 5C is an exploded perspective view of a handle assembly for the catheter of FIG. 5A, according to some embodiments;

FIG. 5D is an exploded perspective view of a deflection assembly for the catheter of FIG. 5A, according to some embodiments;

FIG. 5E is a perspective view of a part of a wire controller of the assembly of FIG. 5D, according to some embodiments;

FIG. 6A is a longitudinal cross-section view of a handle assembly of the catheter of FIG. 5A, according to some embodiments;

FIG. 6B is a longitudinal cross-section view of a deflection assembly enlarged and isolated from the rest of the handle assembly of FIG. 6A;

FIG. 6C is a longitudinal cross-section view of the same handle assembly shown in FIG. 6A, but in which the deflection assembly thereof has been moved;

FIG. 6D is a longitudinal cross-section view of the deflection assembly enlarged and isolated from the rest of the handle assembly of FIG. 6C;

FIG. 7 is a longitudinal cross-section view of a portion of the catheter of FIG. 4A modified to include a wire controller of the deflection assembly of FIG. 5D, according to yet further embodiments of the present invention;

FIG. 8A is a longitudinal cross-section view of a portion of a catheter, according to some additional embodiments;

FIG. 8B is a longitudinal cross-section view of the catheter of FIG. 8A, in which a deflection assembly thereof has been moved in a proximal direction, according to some embodiments; and

FIG. 8C is a longitudinal cross-section view of the catheter of FIG. 8A, in which a portion of the deflection assembly thereof has been moved distally from the position shown in FIG. 8B, according to some embodiments.

### DETAILED DESCRIPTION

The following detailed description is exemplary in nature and is not intended to limit the scope, applicability, or configuration of the invention in any way. Rather, the following description provides practical examples, and those skilled in the art will recognize that some of the examples may have suitable alternatives.

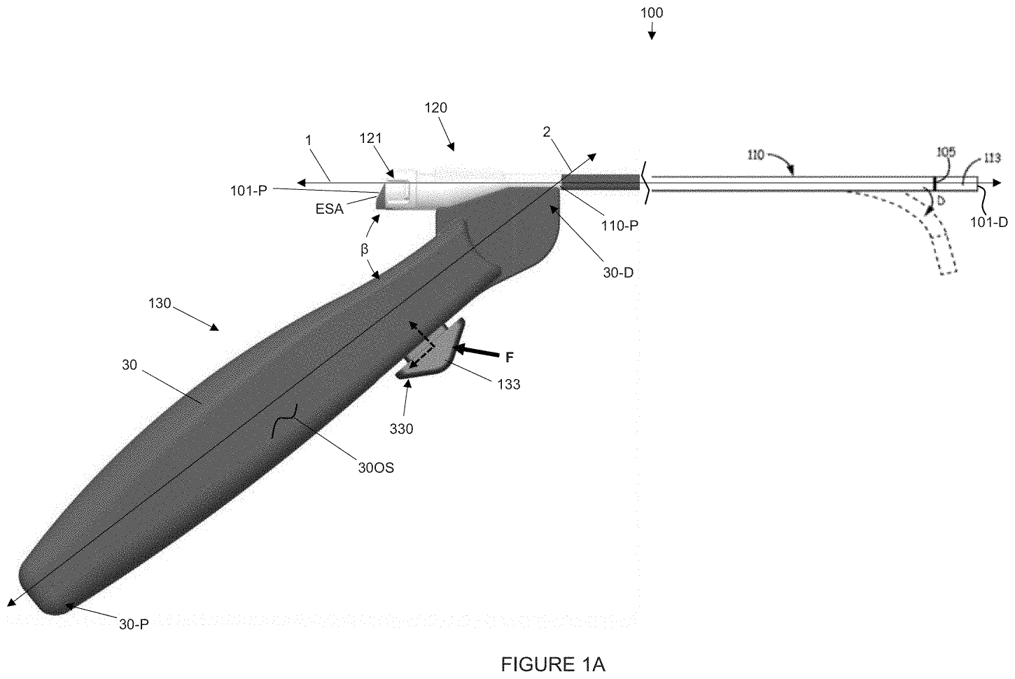

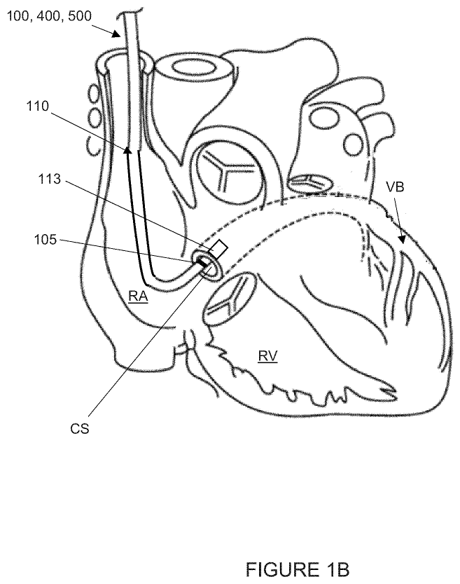

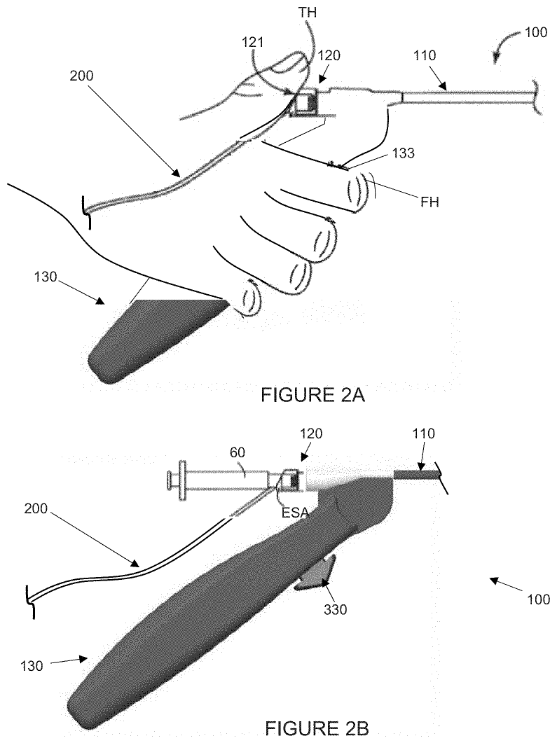

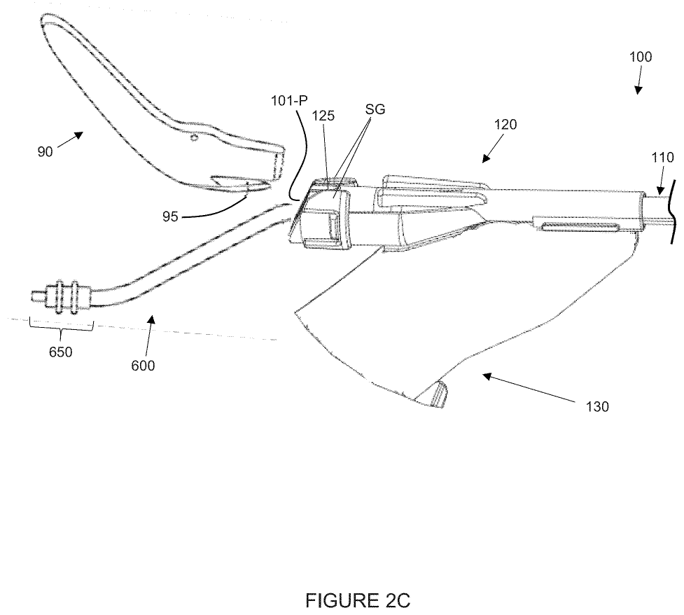

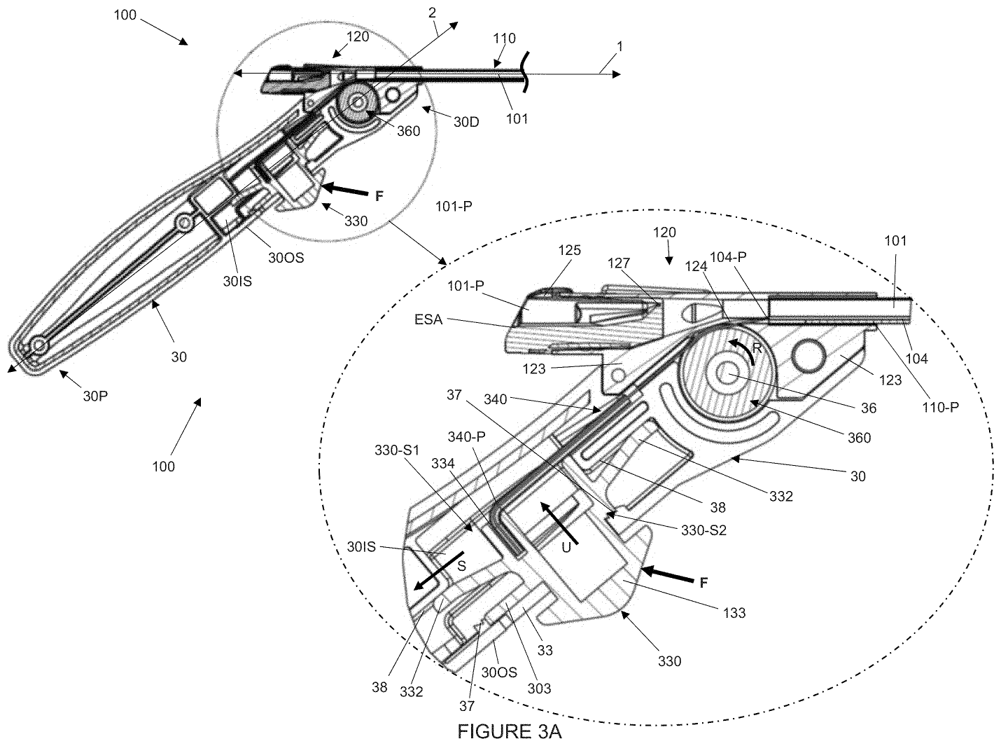

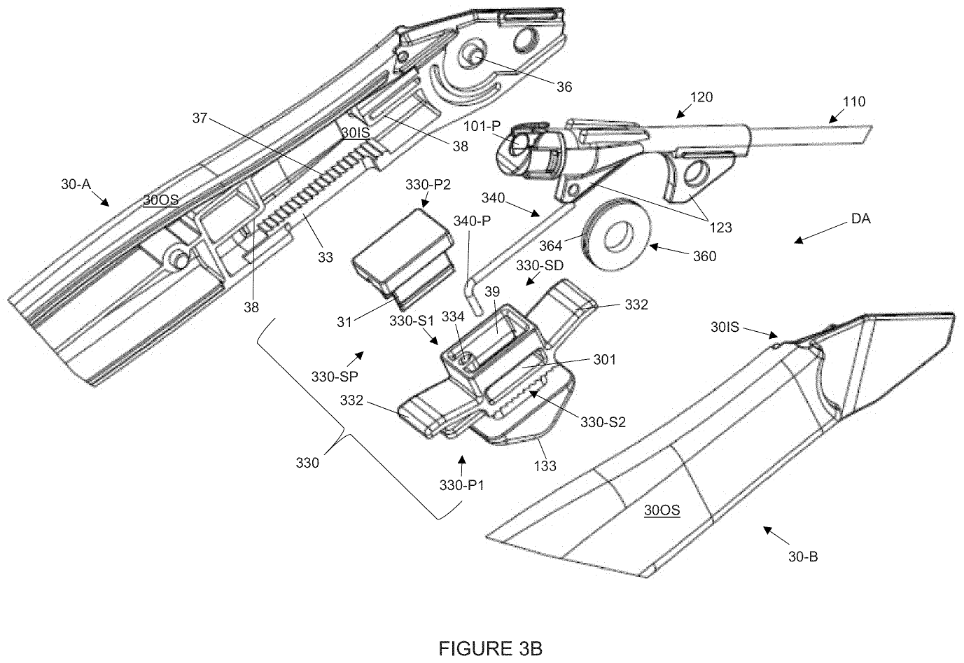

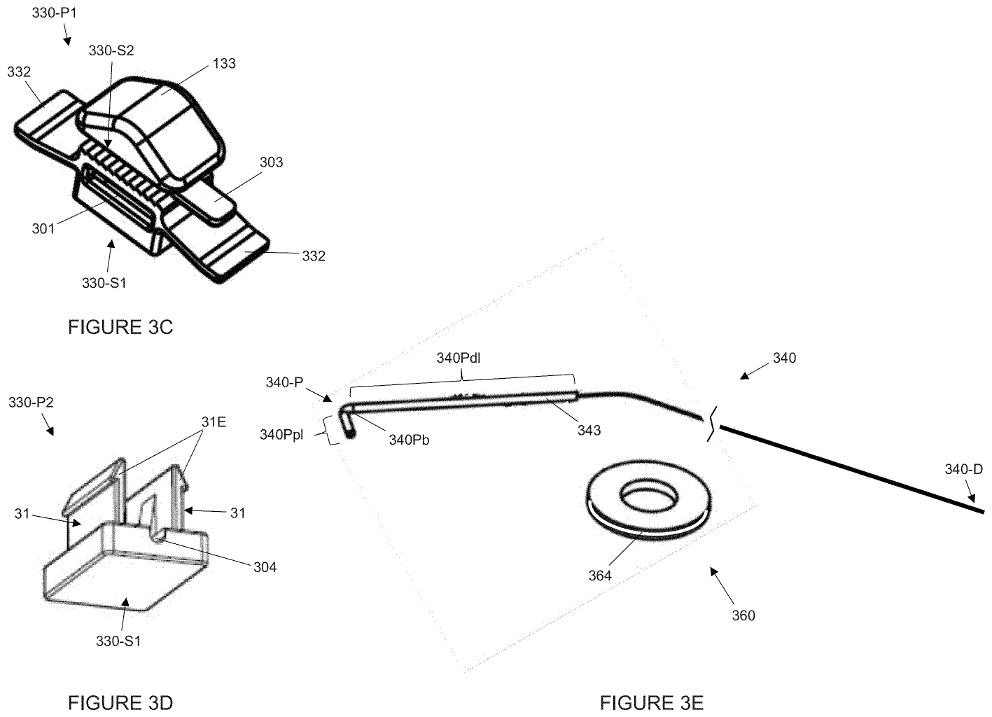

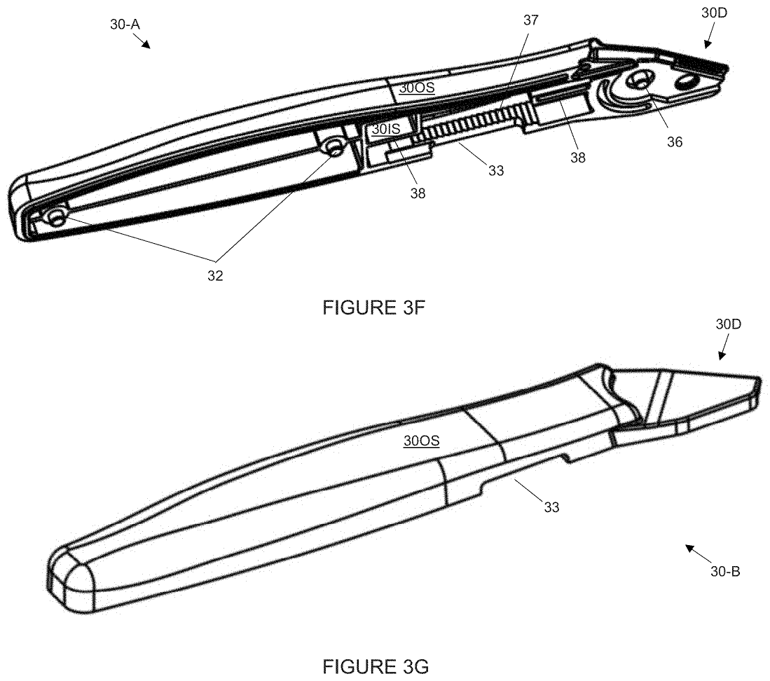

FIG. 1A is a plan view of a deflectable shaft catheter 100, according to some embodiments of the present disclosure. FIG. 1A illustrates catheter 100 including a deflectable shaft 110, a hub 120 coupled to a proximal end 110-P of shaft 110, and a handle 130 coupled to hub 120 and extending laterally therefrom. According to the illustrated embodiment, shaft 110 and hub 120 define an elongate delivery lumen 101 of catheter 100, which can be seen in the cross-section view of FIG. 3A. A proximal opening 101-P of lumen 101 is shown being defined by a proximal end 121 of hub 120, and a distal opening 101-D of lumen 101 is shown being formed at a distal end 113 of shaft 110. Thus, delivery lumen 101 allows passage of a medical device therethrough, for example, a medical electrical lead. FIGS. 1A and 3A further illustrate a first longitudinal axis 1 of catheter 100 being defined by the extent of delivery lumen 101 between proximal opening 101-P and distal opening 101-D, and a second longitudinal axis 2 of catheter 100 being defined by the lateral extent of handle 130. A crossing of second axis 2 with first axis 1 forms an acute angle θ between handle 130 and hub 120, which is designated in FIG. 1A. The orientation of handle 130 relative to shaft 110 and hub 120, which is defined by angle β, facilitates a handling of catheter, for example, as described below in conjunction with FIGS. 2A-C.

Referring to FIG. 3A, shaft 110 further includes an elongate wire lumen 104 that extends alongside delivery lumen 101 and in which an elongate wire 340 extends. A distal end 340-D of wire 340 (shown in FIG. 3E) is anchored in proximity to distal end 113 of shaft 110, for example, being coupled thereto by a band 105 mounted within a wall of shaft 110, as indicated in FIG. 1A and according to construction methods known in the art. Wire 340 extends proximally from band 105 to exit wire lumen 104 at a proximal opening 104-P thereof (shown in FIG. 3A), and further extends within a channel 124 of hub 120 (communicating with wire lumen 104 and with an interior of handle 130), so that a proximal end 340-P of wire 340 is located within handle 130, as seen in FIG. 3A. According to an exemplary embodiment, catheter shaft 110, constructed from one or more medical grades of a polyether block amide (e.g., PEBAX®), may be the same as that employed by the Medtronic Attain™ Deflectable Catheter.

With further reference to FIGS. 1A and 3A, handle 130 is shown having a wire controller 330 integrated therein. An operator interface 133 thereof protrudes from an outer surface 300S of a shell 30 of handle 130. Wire controller 330 and the aforementioned elongate wire 340, which is coupled thereto, may be part of a deflection assembly DA of catheter 100, which is best seen in the exploded view of FIG. 3B. The portion of wire controller 330, according to some embodiments, which extends within handle shell 30 is described below in conjunction with FIGS. 3A-D. According to the illustrated embodiment, when an operator applies a force to interface 133, per arrow F (FIGS. 1A and 3A), wire 340 is moved proximally within wire lumen 104 to deflect shaft 100 in proximity to distal end 113, for example, per arrow D (FIG. 1A). The orientation of handle 130 according to the aforementioned acute angle β can facilitate single-handed manipulation of wire controller 330. FIG. 1B is a schematic that depicts distal end 113 of catheter shaft 110 having been introduced by an operator into a patient's venous system, for example, via a subclavian stick, and positioned within a coronary sinus CS of the patient's heart, the positioning having been facilitated by the deflection of shaft 110. The illustrated position of shaft distal end 113 is suitable for delivering a medical device or other medical agent into the patient's coronary venous system through catheter lumen 101, according to an exemplary method. Alternatively, shaft distal end 113 may be positioned, via shaft 110 deflection, adjacent to a wall of a right ventricle RV or a right atrium RA of the patient's heart to deliver a medical device or agent thereto.

To enhance the handling of catheter 100, for example, as described in conjunction with FIGS. 2A-C, a particularly suitable construction of hub 120, for example, like that of a proximal section of a catheter described in commonly assigned U.S. Pat. No. 8,734,398 to Drake et al., which is hereby incorporated by reference, may be employed in combination with handle 130. FIGS. 2A-B are schematics illustrating how catheter 100 may be handled for maneuvering distal end 113 of catheter shaft 110 into proximity with a target implant site within a patient's body; and FIG. 2C is a schematic illustrating how catheter 100 may be removed from the patient's body.

FIG. 2A is a schematic showing a hand of the operator handling catheter 100, when an elongate tool 200, such as a guide wire or a sub-selecting catheter, extends within catheter delivery lumen 101, according to some methods. FIG. 2A illustrates a thumb TH of the operator's hand positioned over delivery lumen proximal opening 101-P through which tool 200 extends, while a forefinger FH of the operator's hand is positioned over operator interface 133 of wire controller 330 to engage therewith and apply the above-described force that deflects catheter shaft 110. After, and/or while, the operator deflects shaft 110, via wire controller 330, to orient distal opening 101-D of lumen 101 toward the target implant site, the operator may advance a distal end of elongate tool 200 beyond delivery lumen distal opening 101-D. Tool 200 may thus gain access in closer proximity to the target implant site, for example, in one of the branches VB of the patient's coronary venous system (FIG. 1B), that catheter shaft distal end 113 cannot reach. With reference back to FIG. 1A, according to some embodiments, hub proximal end 121 has a tapered profile that orients delivery lumen proximal opening 101-P toward the operator's thumb TH when the fingers of the operator's hand grasp around handle 130 as shown in FIG. 2A. Hub proximal end 121 may further include a relatively soft inner surface that is exposed by the tapered profile and that forms an exposed sealing area ESA, as designated in FIGS. 1A and 3A. Thus, the operator can hold thumb TH of one hand over proximal opening 101-P while manipulating wire controller 330 with forefinger FH of the same hand, as depicted in FIG. 2A, and while advancing tool 200 with the other hand (not shown), so that thumb TH creates a kind of seal with exposed sealing area to prevent excess leakage, or backflow, of blood and/or other fluids out through delivery lumen proximal opening 101-P. Alternately, or in addition, according to some methods, the operator may join an injection syringe 60 to catheter 100, as depicted in the schematic of FIG. 2B, while manipulating tool 200 to position the distal end thereof in closer proximity to the target implant site. Syringe may be used to inject a saline flush through lumen 101, and/or to inject a radiopaque dye through lumen 101 for fluoroscopic visualization of anatomy, for example, branches VB of the patient's coronary venous system (FIG. 1B) distal to distal end 113 of catheter shaft 110, while manipulating tool 200. A standard tapered tip (e.g. slip-tip configuration) of syringe 60 fits alongside tool 200 within proximal opening 101-P formed by hub proximal end 121 and forms a seal with exposed sealing area ESA to prevent the aforementioned backflow.

Once the operator has positioned the distal end of tool 200 at the target implant site, a medical device, such as a medical electrical lead 600, may be advanced over tool 200 and through lumen 101 of catheter 100, for example, as shown in the schematic of FIG. 2C, according to some methods. Alternately, in instances where distal end 113 of catheter shaft 110 can be positioned in close proximity to the target implant site, lead 600 may be advanced through lumen 101 without the need for tool 200. With reference to FIG. 3A, hub 120 may include an internal seal zone 127 sized for sealing around lead 600, for example, as described in the above-referenced U.S. Patent '398. FIG. 2C illustrates a slitting tool 90 positioned for removing catheter 100 from around lead 600 once lead has been implanted. According to the illustrated embodiment, hub 120 includes a relatively thin wall section 125, which extends distally from lumen proximal opening 101-P and is located opposite from handle 130, so that a blade 95 of slitting tool 90 can engage therewith to slit through hub 120 and catheter shaft 110 as catheter 100 is pulled proximally relative to the implanted lead 600. Such a removal process, which is known in the art, may be necessary in the illustrated case because a proximal connector terminal 650 of lead 600 is too large to fit within catheter lumen 101. FIG. 2C further illustrates hub 120 including a pair of outward protruding ridges SG extending along either side of relatively thin wall section 125, which may help to guide slitting tool 90. Wall section 125 may have a thickness comparable to that of catheter shaft 110 (e.g., less than about 0.012 inch) and may be part of a relatively rigid shell of hub 120 into which a relatively soft portion, which forms exposed sealing area ESA and seal zone 127, is fitted, for example, via a two shot molding process known to those skilled in the art. The relatively rigid portion of hub 120 may constructed from medical grade polypropylene, such as 6523 Pro-fax available from LyondellBasell Industries, or polyether block amide, such as PEBAX® 7033 available from Arkema; and the relatively soft portion of hub 130 may be formed from a medical grade Liquid Silicone Rubber, such as CLS2000 LSR available from Momentive, or a thermoplastic elastomer, such as Dynaflex™ G-6703 TPE (available from GLS Corporation), or Medalist® MD-100 TPE (available from Teknor Apex).

Returning to FIG. 3A, wire controller 330 is shown being formed as a spring biased slider that is mounted within handle shell 30. FIG. 3A illustrates an inner surface 30IS of handle shell 30 including a support surface 38 and an engagement feature 37 formed therein. Shell 30 further includes a slot 33 extending therethrough, between a proximal end 30P and a distal end 30D of shell 30 and from inner surface 30IS to outer surface 300S. A length of slot 33 extends along second longitudinal axis 2. Support surface 38, engagement feature 37, and slot 33 are better seen in the exploded view of FIG. 3B. FIG. 3A further illustrates spring biased slider/wire controller 330 being mounted between support surface 38 and slot 33 so that a first side 330-S1 of slider faces toward support surface 38, a second side 330-S2 faces toward engagement feature 37, and operator interface 133 extends from second side 330-S2 and through slot 33 to protrude from outer surface 300S of shell 30. As alluded to above, proximal end 340-P of elongate wire 340 is shown extending within handle shell 30 where it is coupled to wire controller/slider 330, for example, by extending along slider first side 330-S1 and then bending to extend into a bore 334 of slider 330 via an opening thereof located on first side 330-S1. The function of wire controller/slider 330 with respect to the spring bias thereof is described in greater detail below. According to some embodiments, for example, as shown in FIG. 3B, slider 330 includes first and second parts 330-P1, 330-P2. First part 330-P1 defines the aforementioned first and second sides 330-S1, 330-S2, and second part 330-P2 snap-fits together with first part 330-S1 to capture wire proximal end 340-P therebetween. With reference to FIGS. 3B-D, the snap-fit of slider first and second parts 330-P1, 330-P2 may be embodied by apertures 301 of first part 330-P1 and a pair of opposing elastic legs 31 of second part 330-P2. When opposing elastic legs 31 of second part 330-P2 are pushed into opening 39 of first part 330-P1 (FIG. 3B), they are forced toward one another, and then, when each leg end 31E (FIG. 3D) encounters the corresponding aperture 301 of first part 330-P1, each leg 31 elastically springs back to interlock with the corresponding aperture 301, thereby joining parts 330-P1, 330-P2 together. FIG. 3D further illustrates a channel 304 formed in slider second part 330-P2 to accommodate the portion of wire proximal end 340-P that extends along first side 330-S1 of slider 330.

With further reference to FIGS. 3A-B, deflection assembly DA, of which wire 340 and wire controller/spring biased slider 330 are a part, further includes a rotating guide 360. Rotating guide 360 is shown mounted within shell 30 of handle 130, for example, on a spindle 36 formed therein, between hub channel 124 and wire controller/slider 330. FIG. 3A and FIG. 3E, which is a perspective view of elongate wire 340 and rotating guide 360 isolated from the exploded view of FIG. 3B, illustrate guide 360 including an external groove 364 in which wire 340 extends between channel 124 and slider 330. According to the illustrated embodiment, in addition to routing wire 340 into handle shell 30 and providing strain relief for wire 340, the rotation of guide 360 on spindle 36 can prevent wire 340 from cutting into guide 360 as wire controller/slider 330 moves wire 340 relative to wire lumen 104 to deflect shaft distal end 113 (FIG. 1A). Wire 340 may be a medical grade stainless steel wire having a diameter of about 0.010 inch to 0.012 inch, and, as indicated in FIG. 3E, wire proximal end 340-P is preferably reinforced by a sleeve 343 extending thereabout. Sleeve 343 may be formed from medical grade stainless steel hypo-tube, which is attached to wire proximal end 340-P by any suitable method known in the art, such as crimping, welding, swaging, and/or adhesive bonding. According to the illustrated embodiment, the reinforced portion of wire proximal end 340-P has a pre-formed bend 340Pb extending between a proximal length 340Ppl thereof, which is received in slider bore 334, and a distal length 340Pdl thereof, which extends within slider channel 304 when wire 304 is coupled to slider 330.

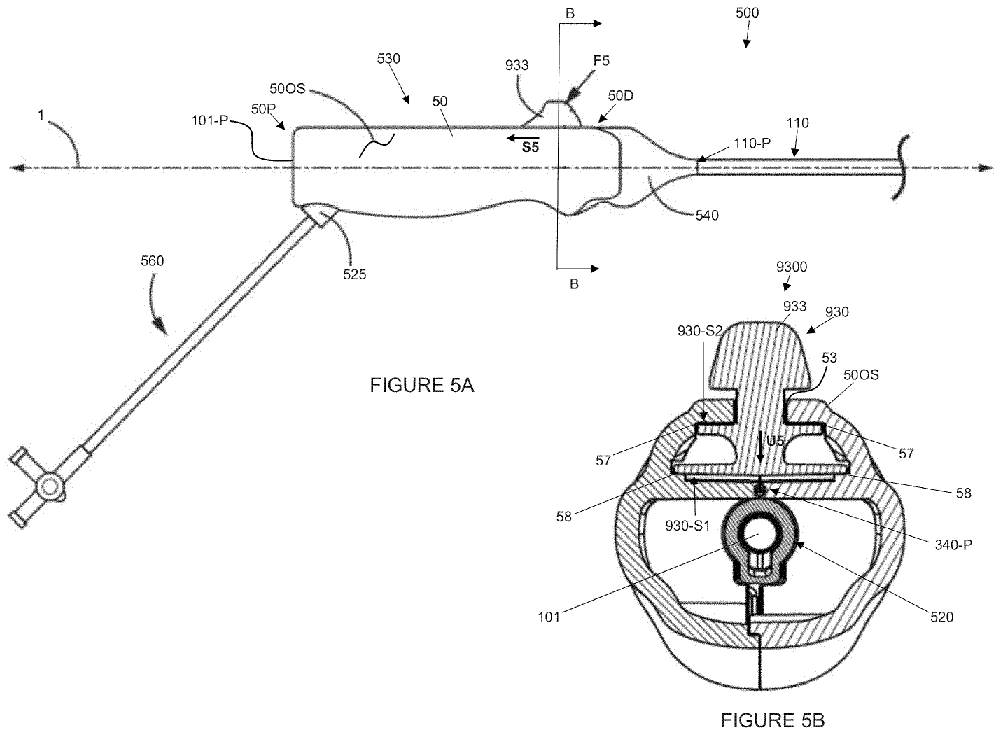

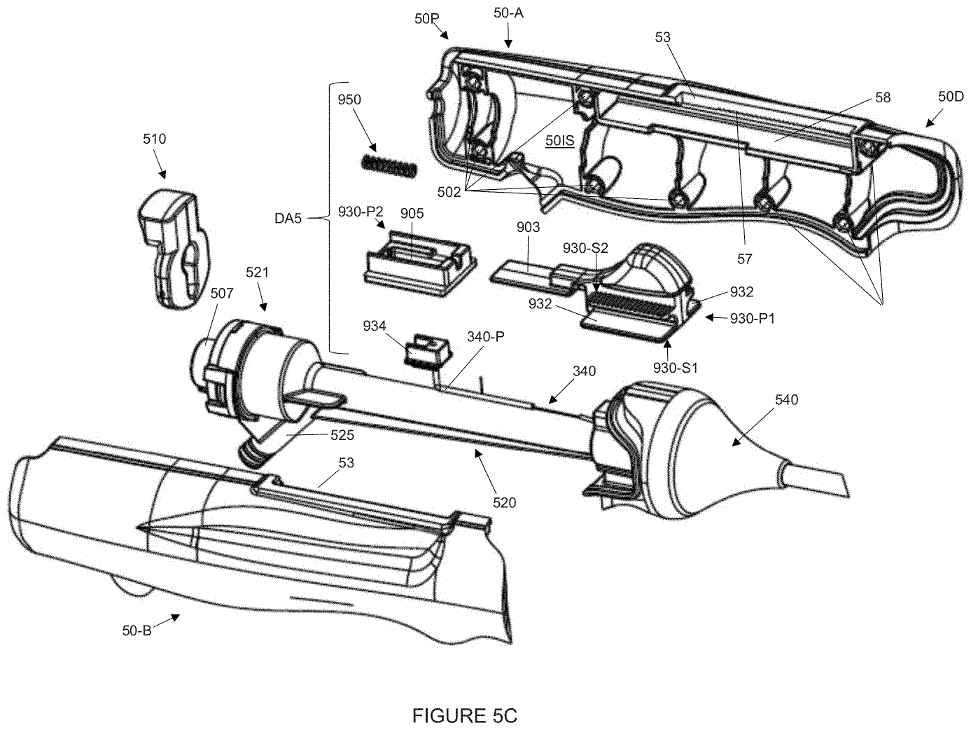

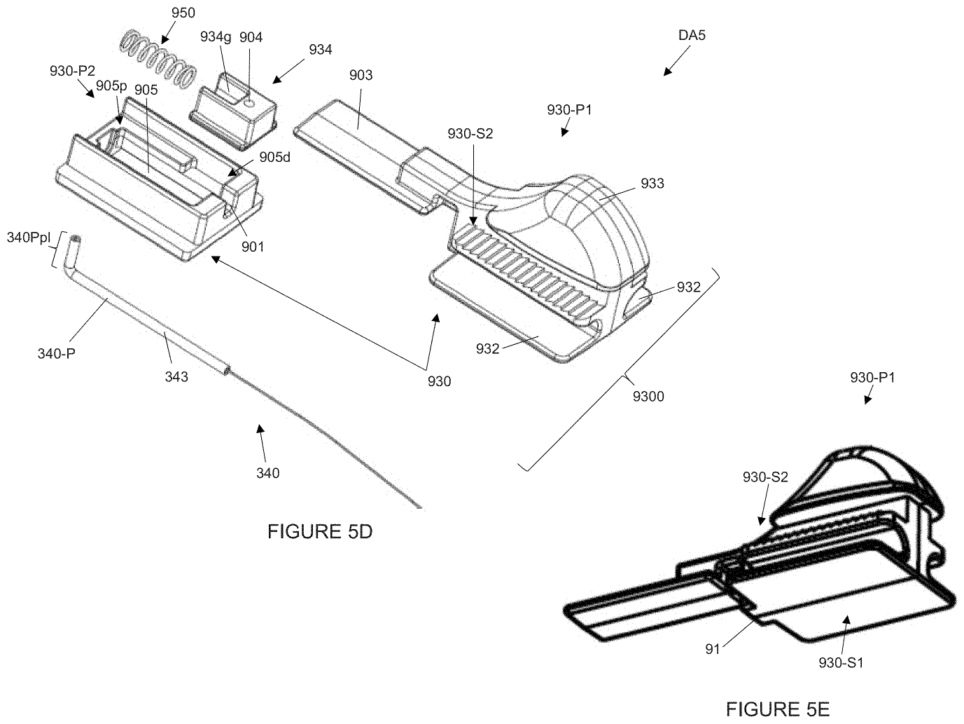

Returning again to FIGS. 3A-B, the significance of the spring bias of wire controller/slider 330 will be described. According to the illustrated embodiment, a portion of slider first side 330-S1 is defined by opposing elastically deformable cantilever beam members 332, and, when slider 330 is mounted in handle shell 30, beam members 332 are supported by shell support surface 38 and provide the spring bias of slider 330. When the mounted slider 330 is relaxed according to the spring bias thereof, second side 330-S2 of slider 330 interlocks with engagement feature 37 of handle shell 30, as illustrated in FIG. 3A. In FIG. 3B (FIGS. 3C and 3F too), slider second side 330-S2 and shell engagement feature 37 are each shown having at least one row of teeth configured to mate/interlock with one another, according to some embodiments. (Although only one row of teeth can be seen for shell 30, formed in a first part 30-A thereof, and for slider 330, it should be appreciated that another row for each extends on the other side of slot 33.) The spring biased mating or interlocking of slider 330 with shell engagement feature 37 prevents movement of slider 330 along the length of slot 33. But, when the operator applies the aforementioned force to interface 133 of slider 330, along the illustrated vector of arrow F, a first component of the force vector F, per arrow U, causes slider beam members 332 to deform against the spring bias thereof so that slider second side 330-S2 moves away from handle shell engagement feature 37 and no longer interlocks therewith, and a second component of the force vector F, per arrow S, causes slider 330 to move proximally along the length of slot 33 with slider first side 330-S1 sliding along support surface 38. With reference to FIGS. 3A and 3C, slider first part 330-P1 further includes an extension 303 for covering an exposed length of slot 33 proximal to operator interface 133. As described above, the movement of wire controller/slider 330 in the proximal direction pulls wire 340 proximally within wire lumen 104, which causes catheter shaft 110 to deflect at distal end 113 thereof (FIG. 1A). An operator can maintain a given degree of deflection in shaft 110, for example, like that illustrated in the schematic of FIG. 1B, without having to maintain the applied force, for when the operator releases the applied force, spring biased slider 330 moves back toward engagement feature 37 and interlocks therewith. To cause shaft distal end 113 to move back toward an un-deflected state, the operator may apply a force to operator interface 133 along a vector that has a first component per arrow U, but has a second component in a direction opposite to arrow S, thereby actively pushing wire 340 distally with wire controller/slider 330, or, due to release from engagement feature 37, allowing shaft distal end 113 to relax back toward the un-deflected state. In some alternate embodiments, for example, as described below in conjunction with FIGS. 5A-8C, a wire controller may include an anti-buckle return mechanism. Such a mechanism can prevent the operator from forcing wire 340 against an opposing force imposed on shaft distal end 113, for example, by surrounding anatomy within the patient's body, because such an opposing force could put undue stress on the coupling between band 105 and wire distal end 340-D.

According to an exemplary embodiment, a thickness of each slider cantilever beam member 332 may be about 0.050 inch; and, in some preferred embodiments, slider first part 330-P1 is injection molded from a medical grade, living-hinge type plastic that enhances the above-described spring function of beam members 332, for example, a nylon or a polyether block amide (e.g., PEBAX®), either of which may be glass-filled, or a polycarbonate. Slider second part 330-P2 may also be injection molded from the same plastic as first part 330-P1, and rotating guide 360 may be formed from a suitable medical grade plastic, such as an acrylonitrile butadiene styrene (ABS), a polycarbonate, a nylon, or a polyether block amide, according to some embodiments.

With further reference to FIG. 3B, handle shell 30 is shown divided into first and second parts 30-A, 30-B, which can be assembled together around deflection assembly DA and around flange features 123 of hub 120 to secure handle 130 to shaft 110. Each flange feature 123 is shown having a bore extending therethrough with which a corresponding bore of handle shell first part 30-A, located in proximity to distal end 30D, can be aligned so that corresponding protruding features of handle shell second part 30-B, also located in proximity to distal end 30D, can extend through the bores of flange features 123 and of shell first part 30-A, when first and second parts 30-A, 30-B are assembled together. Handle shell parts 30-A, 30-B can have additional features formed in the inner surface 301S of each and configured for securing the parts together, for example, pin members 32 of first part 30-A, seen in FIG. 3F, that mate in a press-fit with corresponding receptacles of second part 30-B (not shown). According to some exemplary embodiments, handle shell parts 30-A, 30-B are injection molded from a relatively rigid medical grade plastic, such as Acrylonitrile butadiene styrene (ABS), according to methods known in the art.

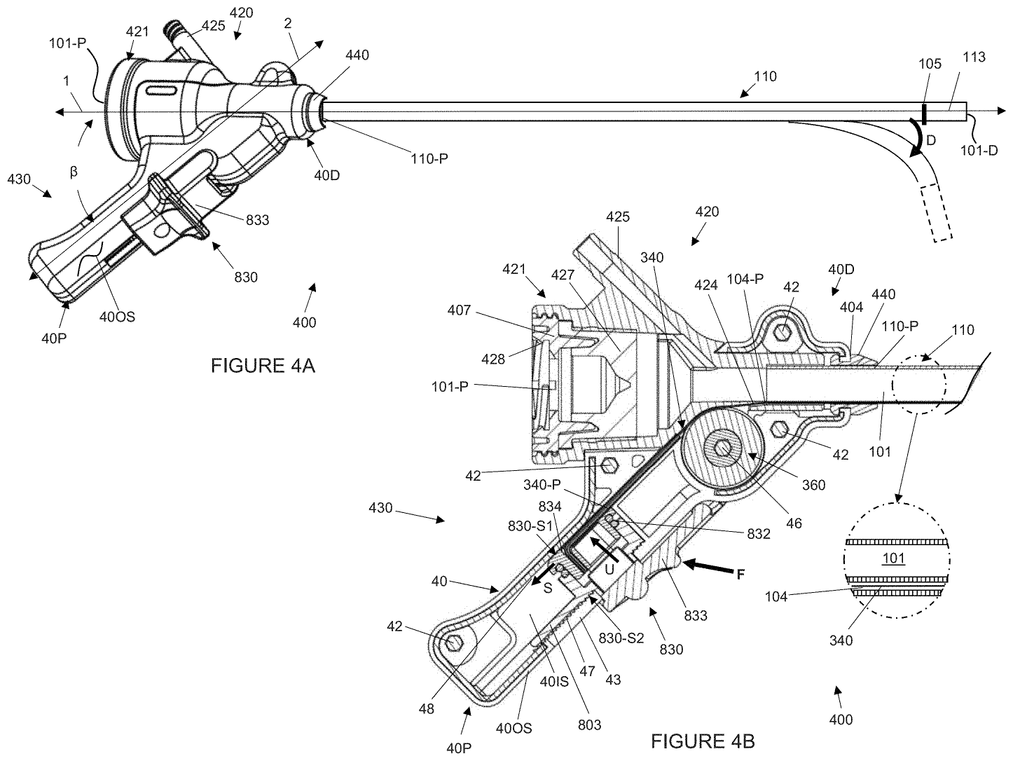

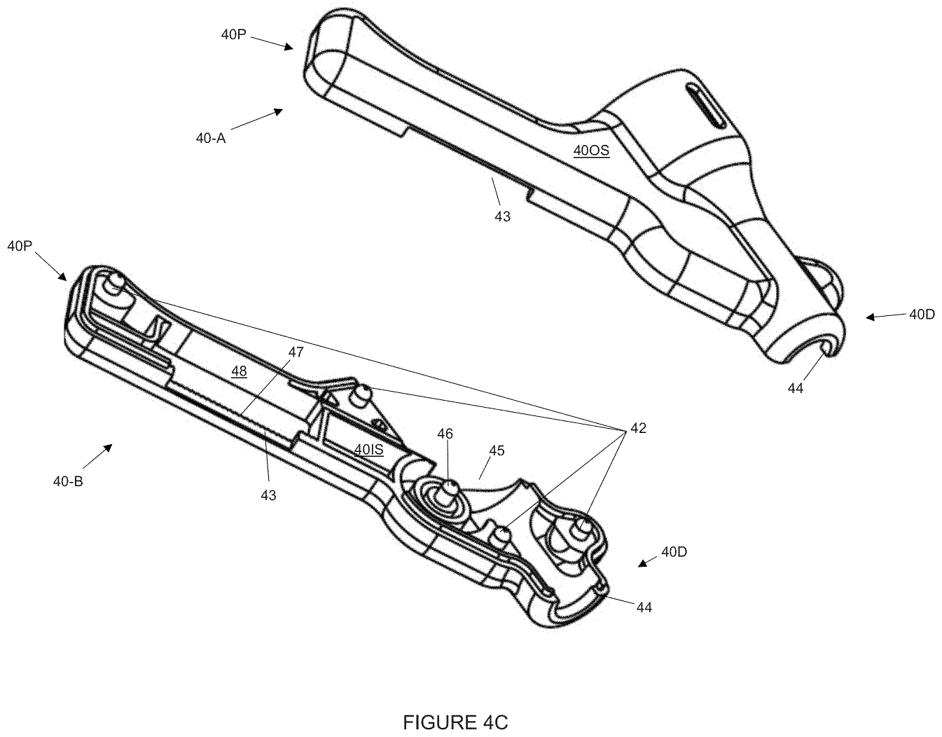

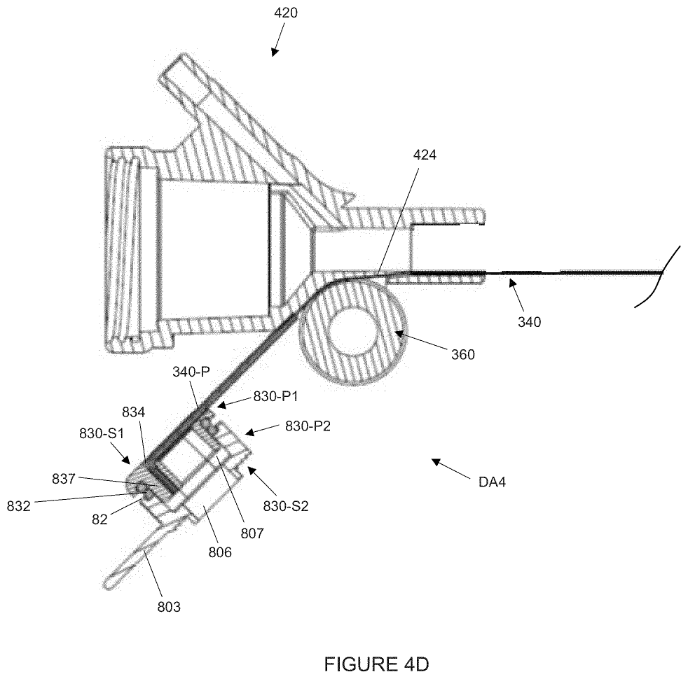

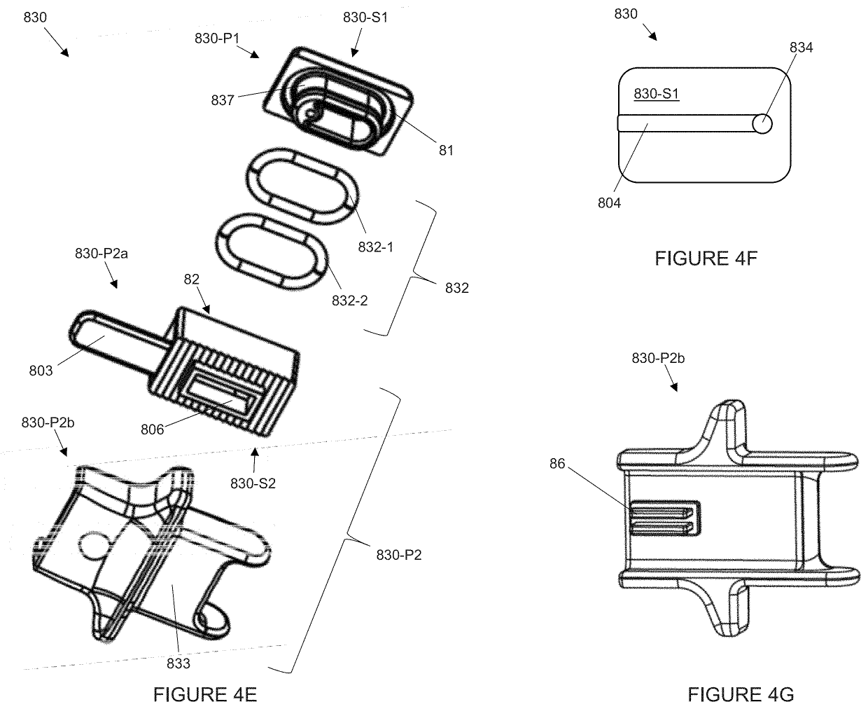

FIG. 4A is a perspective view of a deflectable shaft catheter 400, according to some alternate embodiments of the present disclosure. FIG. 4B is a longitudinal cross-section view with an enlarged detail view of a portion of catheter 400, according to some embodiments. FIGS. 4A-B illustrate catheter 400 including deflectable shaft 110, like catheter 100, and a hub 420 and a handle 430 coupled to shaft proximal end 110-P. A shell 40 of handle 430 has a distal end 40D extending around hub 420. As in catheter 100, shaft 110 and hub 420 of catheter 400 define elongate delivery lumen 101 with lumen proximal opening 101-P defined by a proximal end 421 of hub 420, and lumen distal opening 101-D formed at distal end 113 of shaft 110. Also, like catheter 100, the extent of delivery lumen 101 defines first longitudinal axis 1 of catheter 400, and the lateral extent of handle 430 defines second longitudinal axis 2 of catheter 400. The crossing of second axis 2 with first axis 1 forms acute angle β between handle 430 and hub 420 (designated in FIG. 4A), which can facilitate handling of catheter 400.

FIG. 4B further illustrates elongate wire lumen 104 extending alongside delivery lumen 101 and elongate wire 340 extending therein, similar to catheter 100, for the deflection of shaft 110, for example, per arrow D. Wire 340 extends proximally from band 105, which anchors wire distal end 340-D to shaft 110, and exits wire lumen 104 at proximal opening 104-P thereof. Wire 340 further extends within a channel 424 of hub 420 (communicating with wire lumen 104 and with an interior of handle 430), so that wire proximal end 340-P is located within handle 430. Similar to deflection assembly DA of catheter 100, a deflection assembly DA4 (FIG. 4D) of catheter 400 includes wire 340 and rotating guide 360. Wire 340 extends within external groove 364 of guide 360 (FIG. 3E), between hub 420 and a wire controller formed by a spring biased slider 830, which is also part of deflection assembly DA4. Wire controller/slider 830 is shown integrated into handle 430 so that an operator interface 833 thereof protrudes from an outer surface 400S of handle shell 40. Wire controller/spring biased slider 830 is described in greater detail below. Handle shell 40 may be divided into first and second parts 40-A and 40-B, which are illustrated in the exploded perspective view of FIG. 4C. In FIG. 4B, guide 360 is shown mounted on a spindle 46, which may be formed in an inner surface 4015 of handle shell 40 as shown in FIG. 4C.

With further reference to FIGS. 4A-C, first and second parts 40-A, 40-B of handle shell 40 may be coupled together around hub 420, shaft proximal end 110-P, and deflection assembly DA4. Hub 420 and rotating guide 360 are shown located in proximity to shell distal end D-40, and wire controller/slider 830 is mounted between a support surface 48 and an engagement feature 47 of shell 40, both formed in inner surface 4015 of shell 40, closer to shell proximal end 40-P. FIGS. 4B-C illustrate a slot 43 formed when handle shell parts 40-A, 40-B are coupled together. Slot 43 extends from shell inner surface 4015 to shell outer surface 400S and has a length that extends along second longitudinal axis 2. With reference to FIG. 4B, the aforementioned operator interface 833 of wire controller/slider 830 extends through slot 43. In FIG. 4C, handle shell second part 40-B is also shown including an opening 45 formed at distal end 40D which provides clearance for a side port 425 of hub 420, as illustrated in FIG. 4B. FIG. 4B further illustrates a valve member 427 being mounted within hub proximal end 421 defining an internal seal zone, for example, similar to seal zone 127 of catheter 100. Valve member 427 is shown being secured in place by a valve cap 407 that has threads 428 formed therein. Threads 428 extend around lumen proximal opening 101-P, for example, for the securement of a dilator tool that extends within lumen 101 upon initial insertion of catheter 400.

FIG. 4C further illustrates handle shell second part 40-B including a plurality of pin members 42 protruding from inner surface 4015, which are configured to mate, for example, in a press-fit, with corresponding receptacles (not shown) formed in inner surface 4015 of shell first part 40-A to secure parts 40-A, 40-B together, according to some embodiments. With reference back to FIGS. 4A-B catheter 400 is shown including a strain relief collar 440 that extends around shaft proximal end 110-P, being retained by handle shell distal end 40D, for example, by engagement of rolled under edges 44 of shell parts 40-A, 40-B within an annular groove 404 formed in an external surface of collar 440. According to some exemplary embodiments, handle shell parts 40-A, 40-B are injection molded from a relatively rigid medical grade plastic, such as Acrylonitrile butadiene styrene (ABS), according to methods known in the art. Furthermore, strain relief collar 440 may be injection molded from a medical grade silicone rubber.

FIG. 4D is a longitudinal section view of hub 420 and deflection assembly DA4, sans operator interface 833, which are isolated from the rest of catheter 400 for clarity in description. FIG. 4D (as well as FIG. 4B) illustrates wire controller/spring biased slider 830 including a first side 830-S1 and a second side 830-S2 opposite first side 830-S1, when mounted within handle shell 40 as described above, slider first side 830-S1 faces toward support surface 48 of shell 40 and slider second side 830-S2 faces toward engagement feature 47 of shell 40. The aforementioned operator interface 833 extends from slider second side 830-S2 and through slot 43, as illustrated in FIG. 4B. As alluded to above, proximal end 340-P of elongate wire 340 is coupled to wire controller/slider 830, for example, by extending along slider first side 830-S1 and then bending to extend into a bore 834 of slider 830 via an opening thereof located on first side 830-S1, which can be seen in the plan view of FIG. 4F. (As in catheter 100, wire proximal end 340-P in catheter 400 preferably includes the aforementioned reinforcing sleeve 343 described above in conjunction with FIG. 3E.) FIG. 4F further illustrates a groove 804 formed in slider first side 830-S1 to receive distal length 340Pdl of wire proximal end 340-P (FIG. 3E) when wire 340 is coupled to slider 830.

With reference back to FIG. 4B, the function of wire controller/slider 830 is similar to that described above for wire controller/slider 330 of catheter 100, but the spring bias of slider 830 is provided by an elastomeric core 832. Core 832 may be sandwiched between first and second parts 830-P1, 830-P2 of slider 830, for example, as described below in conjunction with FIG. 4E. When slider 830 is mounted in handle shell 40 and relaxed according to the spring bias thereof, slider second side 830-S2 interlocks with engagement feature 47 of handle shell 40, as illustrated in FIG. 4B. Like the above-described slider 330 and handle shell 30, second side 830-S2 of slider 830 and shell engagement feature 47 may each have at least one row of teeth configured to mate/interlock with one another, according to some embodiments, for example, as illustrated in FIGS. 4B-C and 4E. (Although only one row of teeth can be seen for shell 40, formed in second part 40-B, it should be appreciated that a row extends on either side of slot 43.) The spring biased mating or interlocking of wire controller/slider 830 with shell engagement feature 47 prevents movement of slider 830 along the length of slot 43. But, with further reference to FIG. 4B, when the operator applies a force to interface 833 of slider 830, along the illustrated vector of arrow F, a first component of the force vector F, per arrow U, causes elastomeric core 832 to deform against the spring bias thereof so that slider second side 830-S2 moves away from handle shell engagement feature 47 and no longer interlocks therewith, and a second component of the force vector F, per arrow S, causes slider 830 to move proximally along the length of slot 43 with slider first side 830-S1 sliding along support surface 48. With reference to FIGS. 4B, 4D and 4E, wire controller/slider 830 further includes an extension 803 for covering an exposed length of slot 43 proximal to operator interface 833. As described above, the movement of wire controller/slider 830 in the proximal direction pulls wire 340 proximally within wire lumen 104, which causes catheter shaft 110 to deflect at distal end 113 thereof (FIG. 4A). An operator can maintain a given degree of deflection in shaft 110, for example, like that illustrated in the schematic of FIG. 1B, without having to maintain the applied force, for when the operator releases the applied force, spring biased slider 830 moves back toward engagement feature 47 of handle shell 40 and interlocks therewith. To cause shaft distal end 113 to move back toward an un-deflected state, the operator may apply a force to operator interface 833 along a vector that has a first component per arrow U, but has a second component in a direction opposite to arrow S, thereby actively pushing wire 340 distally with wire controller/slider 830, or, due to release from engagement feature 47, allowing shaft distal end 113 to relax back toward the un-deflected state. As previously mentioned, in an alternate embodiment of a wire controller, which is described below, an anti-buckle return mechanism is employed.

FIG. 4E is an exploded perspective view of wire controller/spring biased slider 830, according to some embodiments. FIG. 4E illustrates slider 830 including the aforementioned first and second parts 830-P1, 830-P2 and first and second elastic ring members 832-1, 832-2, which form the aforementioned elastomeric core 832. In some alternate embodiments, a single elastic ring member or band/pad of a suitable thickness and elasticity may be employed in lieu of first and second members 832-1, 832-2. FIG. 4E further illustrates slider first part 830-P1 including a boss 837, which is sized to fit within a bore 807 of second part 830-P2 (seen in FIG. 4D), for example, in a slip-fit, and around which elastic core 832, for example, stacked together elastic ring members 832-1, 832-2, may be mounted so that core 832 abuts a surface 81 of slider first part 830-P1. With reference to FIG. 4D, when ring members 832-1, 832-2 are thus mounted, and boss 837 of first part 830-P1 is fit into bore 807 of second part 830-P2, elastic ring members 832-1, 832-2 are sandwiched between surface 81 of first part 830-P1 and an opposing surface 82 of second part 830-P2 to provide the spring bias of wire controller/slider 830. Slider first part 830-P1 may be formed from a suitable medical grade plastic, such as a nylon, polypropylene or polyether block amide, and elastic ring members 832-1, 832-2 may be formed from medical grade silicone rubber.

With further reference to FIGS. 4D-E, slider second part 830-P2 may be formed from first and second components 830-P2a, 830-P2b. Second component 830-P2b, which forms operator interface 833, is configured for a snap-fit with first component 830-P2a. FIG. 4G is a perspective view of second component 830-P2b showing a side opposite to that of operator interface 833. FIG. 4G illustrates a pair of columnar members 86 protruding from the side, which are configured for the snap-fit within an orifice 806 of first component 830-P2a that is located on slider second side 830-S2 (FIG. 4E). Each of first component 830-P2a and second component 830-P2b (operator interface 833) of slider second part 830-P2 may be formed from a suitable medical grade plastic, such as a nylon, polypropylene, or polyether block amide.

FIG. 5A is a plan view of a portion of a deflectable shaft catheter 500, according to some additional embodiments of the present invention; and FIG. 5B is a cross-section view through section line B-B of FIG. 5A, according to some embodiments. FIG. 5A illustrates catheter 500 including deflectable shaft 110, similar to catheters 100, 400, but a handle 530 of catheter 500, which is coupled to shaft proximal end 110-P, extends lengthwise along first longitudinal axis 1, in-line with shaft 110. As described above and shown in FIGS. 3A and 4B, deflectable shaft 110 includes elongate wire lumen 104 in which elongate wire 340 extends. In catheter 500, wire 340 is part of a deflection assembly DA5, which is shown in FIGS. 5C-D, wherein wire distal end 340-D is coupled to band 105 in proximity to distal end 113 of shaft 110, as in catheters 100, 400. According to the illustrated embodiment, wire proximal end 340-P extends within a shell 50 of handle 530 and is coupled to a wire controller 9300, which is mounted within handle 530 and extends through a slot 53 of shell 50 so that an operator interface 933 thereof protrudes from an outer surface 500S of shell 50. Operator interface 933 is part of a portion of wire controller 9300 that is formed by a spring-biased slider 930, which is shown in FIG. 5B. FIG. 5B illustrates slider 930 having a first side 930-S1 facing toward a support surface 58 of handle shell 50, and second side 930-S2 facing toward an engagement feature 57 of handle shell 50, wherein operator interface 933 extends from second side 930-S2. With reference to the exploded perspective view of FIG. 5C, support surface 58 and engagement feature 57 may each be formed in an inner surface 50IS of handle shell 50, and slot 53 extends from shell inner surface 50IS to shell outer surface 500S and along a length of handle shell 50. In the embodiment of FIG. 5C, handle shell 50 is divided into first and second parts 50-A, 50-B. A plurality of pin members (not seen) that protrude from inner surface 50IS of shell second part 50-B may mate, for example, in a press-fit, with a corresponding plurality of receptacles 502 formed in inner surface 50IS of shell first part 50-A to secure parts 50-A, 50-B together around deflection assembly DA5 and a hub 520 of catheter 500, which is described in greater detail below. According to some exemplary embodiments, handle shell parts 50-A, 50-B are injection molded from a relatively rigid medical grade plastic, such as Acrylonitrile butadiene styrene (ABS), according to methods known in the art.

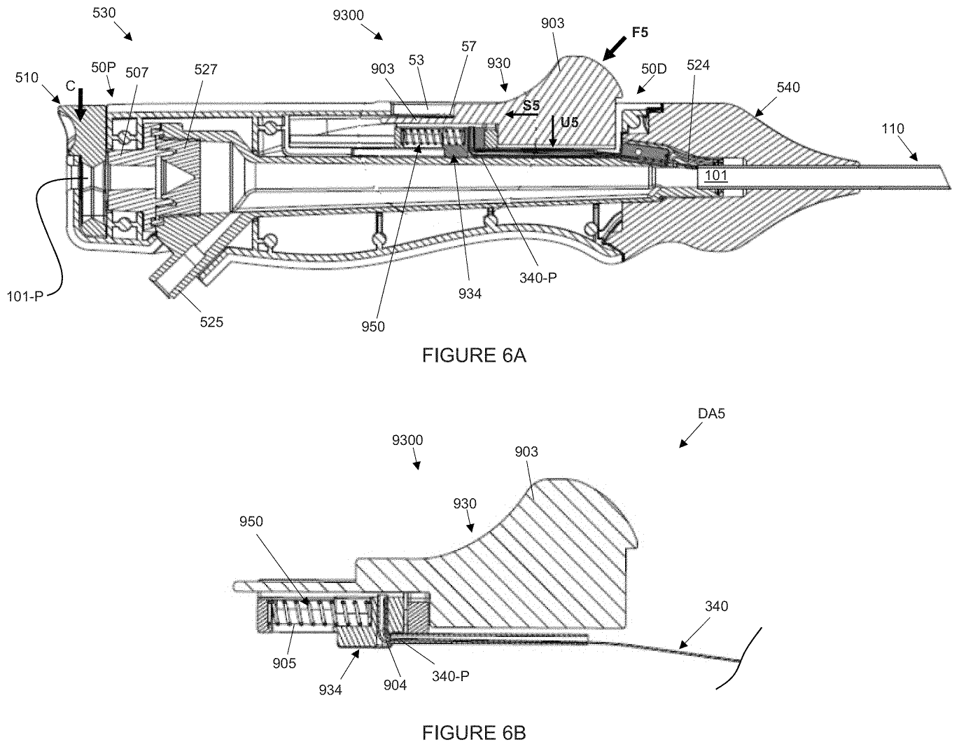

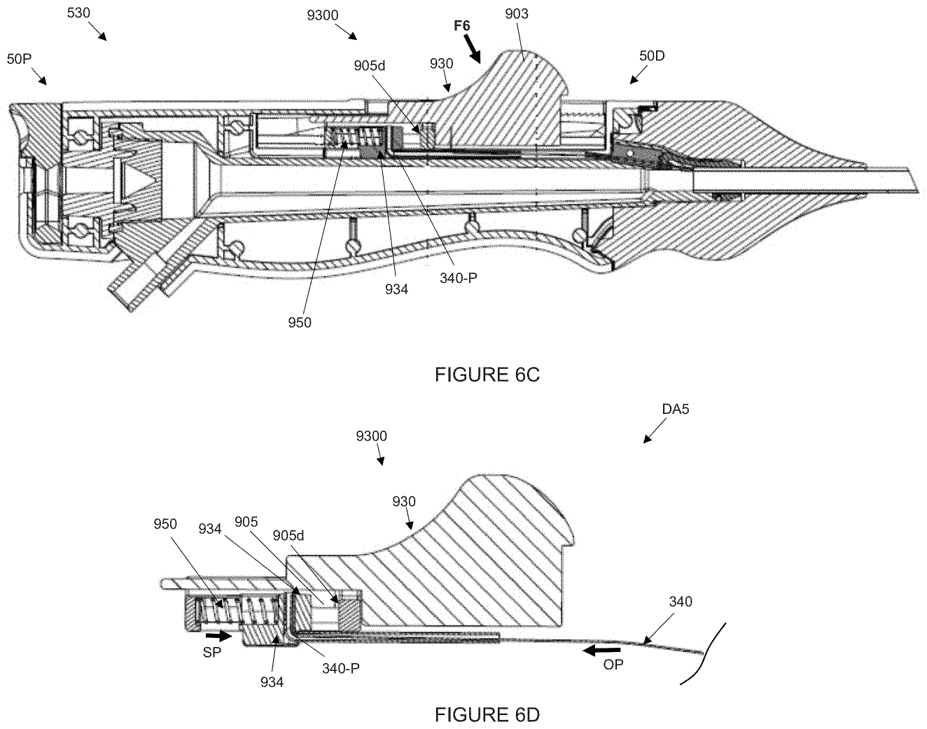

According to the illustrated embodiment, the spring-bias of slider 930 is provided by opposing elastically deformable cantilever beam members 932. Beam members 932 define a portion of slider first side 930-S1 and each extends laterally from slider operator interface 933, in a direction generally orthogonal to longitudinal axis 1 and the length of handle 530, being supported by shell support surface 58 when slider 930 is mounted in handle shell 50. When the mounted slider 930 is relaxed according to the spring bias thereof, slider second side 930-S2 interlocks with engagement feature 57 of handle shell 50, as illustrated in FIG. 5B and in the longitudinal cross-section view of FIG. 6A. FIG. 5C illustrates slider second side 930-S2 and shell engagement feature 57 each having at least one row of teeth configured to mate/interlock with one another, according to some embodiments. (Although only one row of teeth can be seen for shell 50, formed in first part 50-A, and for slider 930, it should be appreciated that another row of teeth for each extends on the other side of slot 53 as is evident with reference to FIG. 5B.) The spring biased mating or interlocking of wire controller slider 930 with shell engagement feature 57 prevents movement of slider 930 along the length of slot 53. But, when the operator applies a force to interface 933 of wire controller slider 930, along the illustrated vector of arrow F5 (FIGS. 5A and 6A), a first component of the force vector F5, per arrow U5 (FIGS. 5B and 6A), causes slider beam members 932 to deform against the spring bias thereof so that slider second side 930-S2 moves away from handle shell engagement feature 57 and no longer interlocks therewith, and a second component of the force vector F5, per arrow S5 (FIGS. 5A and 6A), causes spring biased slider 930 to move proximally along the length of slot 53. As described above for catheters 100, 400, the movement of wire controller 9300 in the proximal direction pulls wire 340 proximally within wire lumen 104, which causes catheter shaft 110 to deflect at distal end 113 thereof. Again, an operator can maintain a given degree of deflection in shaft 110, for example, like that illustrated in the schematic of FIG. 1B, without having to maintain the applied force, for when the operator releases the applied force, spring biased slider 930 moves back toward engagement feature 57 and interlocks therewith. To cause shaft distal end 113 to move back toward an un-deflected state, the operator may apply a force to operator interface 933 along a vector that has a first component per arrow U5, but has a second component in a direction opposite to arrow S5, thereby actively pushing wire 340 distally with wire controller 9300, or, due to release from engagement feature 57, allowing shaft distal end 113 to relax back toward the un-deflected state. The longitudinal cross-section view of FIG. 6C illustrates wire controller 9300 having been moved to push wire 340 according to such a force applied per arrow F6. However, wire controller 9300 further includes a spring member 950 that, when mounted in a cavity 905 of slider 930, forms an anti-buckle return mechanism, as alluded to above. The anti-buckle return mechanism keeps the operator from forcing wire 340 against an opposing force imposed on shaft distal end 113, for example, by the surrounding anatomy, which could put undue stress on the coupling between band 105 and wire distal end 340-D. With further reference to FIG. 5C, slider 930 also includes an extension 903 for covering an exposed length of slot 53 proximal to operator interface 933 of slider 930, for example, as illustrated in FIG. 6A.

FIG. 5C further illustrates spring biased slider 930 of wire controller 9300 including first and second parts 930-P1, 930-P2. First part 930-1 is that seen in FIG. 5B and includes beam members 932, operator interface 933, and extension 903. Second part 930-P2 defines the aforementioned cavity 905 of slider 930, in which spring member 950 is mounted, as described in greater detail below. With reference to the exploded perspective view of deflection assembly DA5 in FIG. 5D, and to the perspective view of slider first part 930-1 in FIG. 5E, slider first and second parts 930-P1, 930-P2 may be joined together by fitting a projection 91 of first part 930-P1 (FIG. 5E) within a notch 901 of second part 930-P2 (FIG. 5D) so that cavity 905 will be located between support surface 58 and engagement feature 57 of handle shell 50, when slider 930 is mounted within shell 50, for example, as illustrated in FIGS. 6A and 6C. With reference to FIG. 5D, cavity 905 is shown having a proximal end 905p and a distal end 905d. When wire controller 9300 is mounted in handle shell 50, cavity proximal end 905p is oriented toward a proximal end 50P of handle shell 50 and cavity distal end 905d is oriented toward a distal end 50D.

According to the illustrated embodiment, wire proximal end 340-P is coupled to wire controller 9300 within cavity 905 of spring biased slider 930, and spring member 950, when mounted in cavity 905, is biased to push wire proximal end 340-P toward distal end 905d thereof, for example, as illustrated in FIGS. 6A-B. FIG. 6B is a longitudinal cross-section view of deflection assembly DA5 enlarged and isolated from the rest of the handle assembly shown in FIG. 6A. With reference to FIGS. 5D and 6B, wire controller 9300 further includes a coupling component 934 which is mounted within cavity 905 between spring member 950 and cavity distal end 905d. Coupling component 934 is shown having a bore 904 into which distal length 340Ppl of wire proximal end 340-P extends and is secured for the coupling of wire proximal end 340-P to wire controller slider 930. FIG. 5D further illustrates wire proximal end 340-P being reinforced by sleeve 343, as described above. With further reference to FIG. 5D, coupling component 934 includes a groove 934g in which spring 950 extends when both are mounted in cavity 905. Slider first and second parts 930-P1, 930-P2 and coupling component may be injection molded from a medical grade plastic, such as a nylon or a polyether block amide (e.g., PEBAX®), either of which may be glass-filled, or a polycarbonate. Spring member 950 is shown as a coiled wire, for example, a medical grade stainless steel wire formed in a coil, but other suitable type of spring element may be employed for spring member 950.

The bias of spring member 950 illustrated in FIGS. 6A-B is governed by a predetermined spring force thereof, which may be established to protect the integrity of the coupling between wire distal end 340-D and band 105 in the aforementioned function of the anti-buckle return mechanism embodied by spring member 950. For example, with reference back to FIG. 1B, if an operator desires to move shaft distal end 113 from the deflected state shown back toward an undeflected state, but distal end 113 is constrained by the surrounding anatomy of the coronary sinus CS, the predetermined spring force of spring member 950 may not be large enough to overcome an opposing force applied along the length of wire 340 by the constraint of the coronary sinus CS. Thus, with reference to the longitudinal cross-section views of FIGS. 6C-D, even though the operator has applied a force to operator interface 933 along the vector of arrow F6, intending to push wire 340 distally and move shaft distal end 113 back toward the undeflected state, the spring force, per arrow SP, of spring member 950 is not sufficient to overcome the opposing force, per arrow OP, applied along the length of wire 340, for example, by the constraining anatomy of the coronary sinus CS (FIG. 1B). With further reference to FIG. 6D, although slider 930 of wire controller 9300 has been moved distally by the operator, the opposing force along wire 340 has prevented spring member 950 from pushing wire proximal end 340-P/coupling component 934 to distal end 905d of cavity 905. According to some alternate embodiments, a wire controller that does not include the anti-buckle return mechanism may be employed by the illustrated handle assembly of catheter 500.

With reference back to FIGS. 5B-C, hub 520 of catheter 500 extends within, and along a length of handle shell 50. According to the illustrated embodiment, a distal end 522 of hub 520 is coupled to shaft proximal end 110-P so that delivery lumen 101 defined by shaft 110 is further defined by hub and extends within handle shell 50 to proximal opening 101-P thereof, which may be located at a proximal end 50P of handle shell 50, as shown in FIG. 6A. FIG. 6A further illustrates hub 520 including a channel 524, which communicates with wire lumen 104 of shaft 110 to allow passage of wire 340 therethrough from shaft 110 to handle 530. FIG. 5C further illustrates hub 520 including a side port 525, which extends through an opening of handle shell 50 to protrude from outer surface 500S thereof, as shown in FIG. 5A. FIG. 5A further illustrates a tube 560 for a flushing assembly that may be joined to side port 525 in some embodiments. With further reference to FIG. 6A, catheter 500 further includes a valve member 527 mounted within hub proximal end 521, a valve cap 507, which secures valve member 527 in place within handle shell 50, and a holding member 510 attached to handle shell proximal end 50P. According to the illustrated embodiment, valve member 527 defines an internal seal zone, for example, similar to seal zone 127 of catheter 100, and holding member 510 is compressible, per arrow C, to narrow lumen proximal opening 101-P, for example, to clamp down on an instrument or device that is inserted within lumen 101. Friction at an interface between holding member 510 and inner surface 501S of handle shell 50 may retain holding member 510 in the compressed/clamping position, and/or the interface may be configured with interlocking detents. Holding member 510 may be wholly formed from a medical grade silicone rubber, for example, having a durometer in a range from 60 to 100 on the Shore A scale. A strain relief element 540 is shown joined to hub distal end 522 in FIGS. 5C and 6A, and, in FIG. 6A, strain relief element 540 is shown joined in interlocking engagement with handle shell distal end 50D. The configurations and constructions of hub 520, valve member 527, valve cap 507, and strain relief element 540, as well as the general integration of these within handle shell 50, may be like that described for a hub and corresponding valve member, valve cap, and strain relief element described in the commonly assigned U.S. Pat. No. 9,937,322 to Drake and Stener, salient portions of which are hereby incorporated by reference.

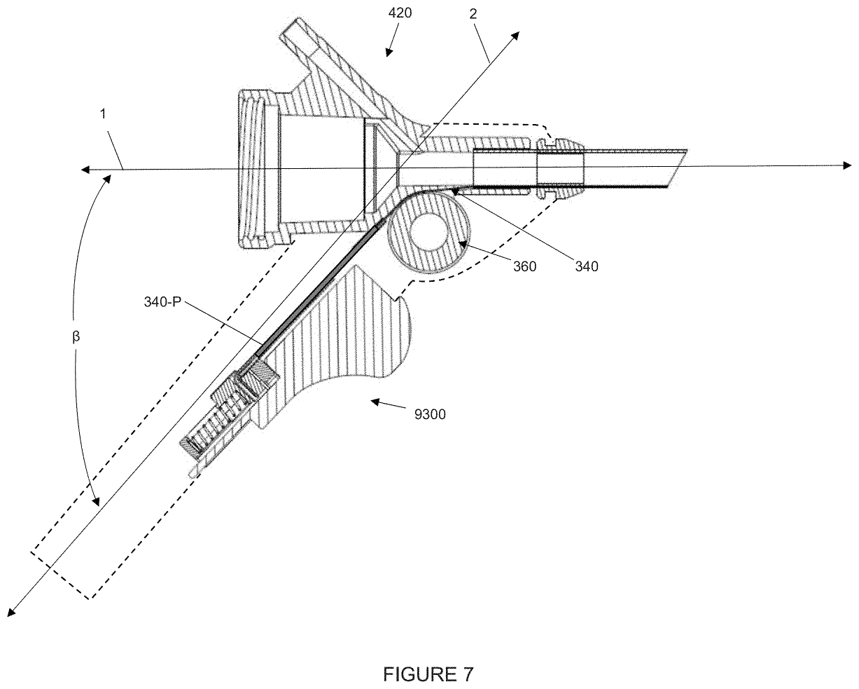

FIG. 7 is a longitudinal cross-section view of a portion of catheter 400 (FIGS. 4A-D) modified to include wire controller 9300 of deflection assembly DA5, in lieu of wire controller/spring biased slider 830, according to yet further embodiments of the present invention. Dashed lines in FIG. 7 illustrate an exemplary form of a handle shell that may be similar to handle shell 40 described above. The embodiment of FIG. 7 combines the anti-buckle return mechanism of catheter 500 with the rotating guide 360 of catheter 400.

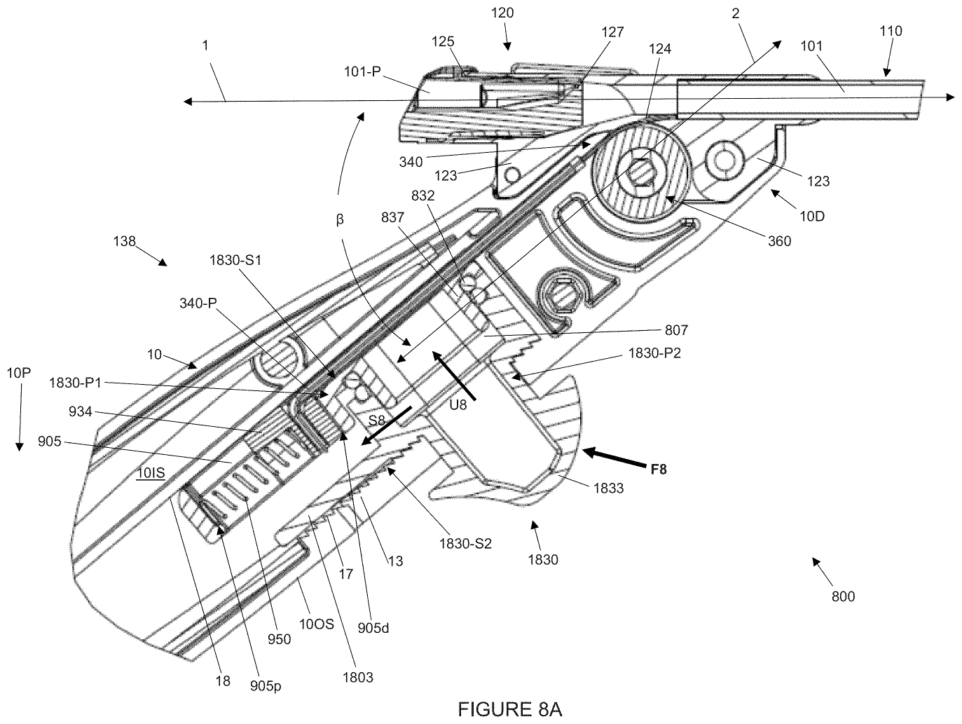

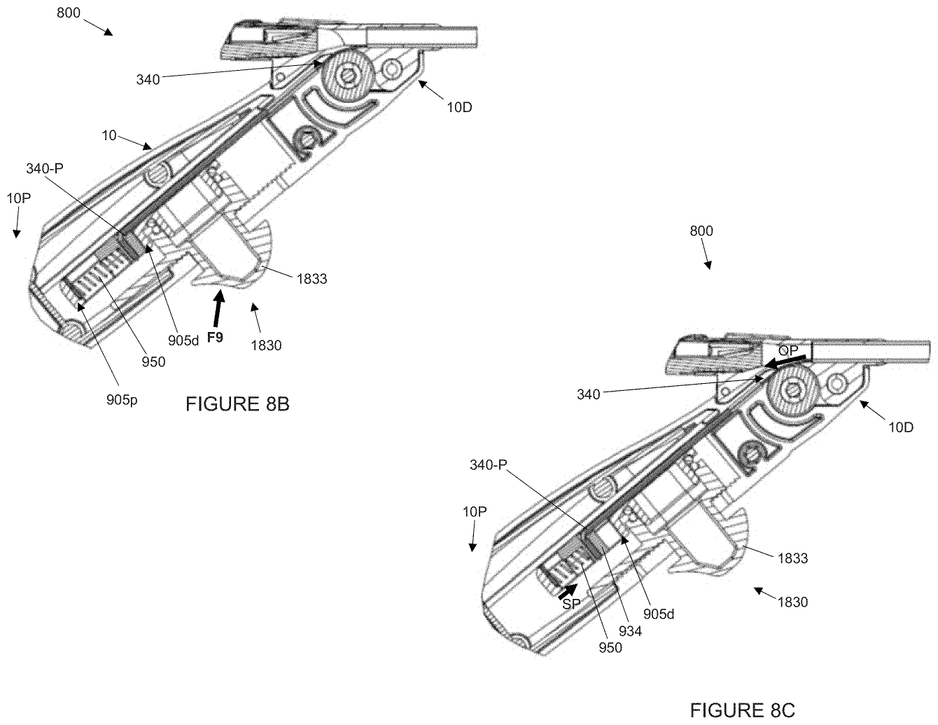

FIG. 8A is a longitudinal cross-section view of a catheter 800, according to some additional embodiments, in which the anti-buckle return mechanism is employed with rotating guide 360. FIG. 8A illustrates catheter 800 including shaft 110 and hub 120, for example, as described above for catheter 100 in conjunction with FIGS. 1A-3A. Catheter 800 is shown including a handle 138 that is oriented at the aforementioned acute angle β, as defined by the axes 1 and 2, to facilitate handling in a similar manner to that described above for catheter 100. FIG. 8A further illustrates a deflection assembly of catheter including the above-described wire 340, rotating guide 360, and a wire controller 1830 to which wire proximal end 340-P is coupled. A shell 10 of handle 138 may be formed in two parts that are fitted together and joined, at a distal end 10D of shell 10, to flange features 123 of hub 120, as described above for handle shell 30, to support rotating guide 360, wire controller 1830, and wire proximal end 340-P in a manner similar to that described above for catheter 100. With reference to FIG. 8A, an inner surface 10IS of handle shell 10 includes a support surface 18 and an engagement feature 17 formed therein, and a slot 13 extending therethrough, from inner surface 10IS to an outer surface 100S. A length of slot 13 extends along second longitudinal axis 2. Handle shell 10 may be injection molded from a relatively rigid medical grade plastic, such as Acrylonitrile butadiene styrene (ABS), according to methods known in the art.

With further reference to FIG. 8A, wire controller 1830 is shown including spring biased slider first and second parts 1830-P1, 1830-P2 between which the above-described elastomeric core 832 is sandwiched to define the spring bias thereof. Like wire controller slider 830 of catheter 400, wire controller 1830 is mounted in handle shell 10 so that a first side 1830-S1 thereof (defined by slider first part 1830-P1) faces toward support surface 18 of shell 10, a second side 1830-S2 thereof (defined by slider second part 1830-P2, which is opposite first side 830-S1, faces toward engagement feature 17 of shell 10, and an operator interface 1833 of wire controller 1830 extends through slot 13 to protrude from shell outer surface 100S. When elastomeric core 832 of wire controller 1830 is relaxed, second side 1830-S2 interlocks with engagement feature 17 of handle shell 10, as illustrated in FIG. 8A. Like the above-described slider 830 and handle shell 40, second side 1830-S2 and shell engagement feature 17 may each have at least one row of teeth configured to mate/interlock with one another, according to some embodiments. (Although only one row of teeth can be seen for shell 10 and slider second part 1830-P2 in the cross-section view, it should be appreciated that a row for each may extend on either side of slot 13 and operator interface 1833, respectively.) The spring biased mating or interlocking of wire controller 1830 with shell engagement feature 17 prevents movement of wire controller 1830 along the length of slot 13, until the operator applies a force to operator interface 1833, for example, along the illustrated vector of arrow F8. Force vector per arrow F8 has a first component, per arrow U8, which causes elastomeric core 832 to deform against the spring bias thereof so that second side 1830-S2 of wire controller 1830 moves away from handle shell engagement feature 17 and no longer interlocks therewith, and a second component, per arrow S8, which causes wire controller 1830 to move proximally along the length of slot 13, with first side 1830-S1 sliding along support surface 18. Second slider component 1830-P2 of wire controller 1830 further includes an extension 1803 for covering an exposed length of slot 13 proximal to operator interface 1833.

According to the illustrated embodiment, and in a similar fashion to above-described wire controllers, the movement of wire controller 1830 in the proximal direction pulls wire 340 proximally, which causes catheter shaft 110 to deflect at distal end 113 thereof. An operator can maintain a given degree of deflection in shaft 110, for example, like that illustrated in the schematic of FIG. 1B, without having to maintain the applied force, for when the operator releases the applied force, wire controller 1830 moves back toward engagement feature 17 of handle shell 10 and interlocks therewith, for example, as illustrated in FIG. 8B. FIG. 8B is a cross-section view of catheter 800, wherein wire controller 1830 has been moved proximally from the position illustrated in FIG. 8A, for example, by the force vector per arrow F8.

With further reference to FIG. 8A in conjunction with FIG. 4E, slider first part 1830-P1, like first part 830-P1 of slider 830, includes boss 837, which is sized to fit within bore 807 of second part 1830-P2, for example, in a slip-fit, and around which elastic core 832, for example, stacked together elastic ring members 832-1, 832-2, may be mounted to be sandwiched between parts 1830-P1, 1830-P2 and thereby provide the aforementioned spring bias. Slider first and second parts 1830-P1, 1830-P2 may be formed from a suitable medical grade plastic, such as a nylon, polypropylene or polyether block amide.

Now with reference to FIG. 5D in conjunction with FIG. 8A, wire controller 1830, like wire controller 9300, further includes spring member 950 mounted in cavity 905 to form the aforementioned anti-buckle return mechanism for the deflection assembly of catheter 800; however, in the illustrated embodiment of wire controller 1830, cavity 905 is defined by slider first part 1830-P1. FIG. 8A illustrates cavity 905 located between support surface 18 and engagement feature 17 of handle shell 10, wherein cavity proximal end 905p is oriented toward a proximal end 10P of handle shell 10, and cavity distal end 905d is oriented toward shell distal end 10D.

According to the illustrated embodiment, spring member 950 is biased to push wire proximal end 340-P, which is coupled to wire controller 1830 within cavity 905, toward cavity distal end 905d. Wire proximal end 340-P may be coupled to spring member 950 via coupling component 934, which is mounted within cavity 905 between spring member 950 and cavity distal end 905d, and which is described above in conjunction with FIG. 5D. As described above, the bias of spring member 950 is governed by the predetermined spring force thereof, which may be established to protect the integrity of the coupling between wire distal end 340-D and band 105 in the aforementioned function of the anti-buckle return mechanism embodied by spring member 950, as described in example above, in conjunction with FIG. 1B. Thus, with reference to the longitudinal cross-section view of FIG. 8C, even though the operator has applied a force to operator interface 1833, for example, along a vector designated with arrow F9 in FIG. 8B, intending to push wire 340 distally and thereby move shaft distal end 113 back toward the undeflected state, the spring force, per arrow SP, of spring member 950 is not sufficient to overcome the opposing force, per arrow OP, applied along the length of wire 340, for example, by the constraining anatomy of the coronary sinus CS (FIG. 1B). With further reference to FIG. 8C, although first and second slider parts 1830-P1, 1830-P2 of wire controller 1830 have been moved distally by the operator's force vector per arrow F9, the opposing force along wire 340 has prevented spring member 950 from pushing wire proximal end 340-P/coupling component 934 to distal end 905d of cavity 905.

### ILLUSTRATIVE EMBODIMENTS

Embodiment 1. A catheter comprising a deflectable shaft, a handle coupled to shaft and extending laterally therefrom and a deflection assembly, the shaft defining a first longitudinal axis of the catheter the deflection assembly comprising an elongate wire and a wire controller, the wire extending along the shaft, the wire having a distal end coupled to the shaft in proximity to the distal end thereof and a proximal end extending within the handle, the wire controller being coupled to the proximal end of the wire within the handle; and the deflection assembly further comprising:

a rotating guide mounted within the handle between the shaft and the wire controller, the guide including an external groove in which the wire extends between the shaft and the wire controller; and

wherein the lateral extent of the handle defines a second longitudinal axis of the catheter, the second axis crossing the first axis to form an angle between the handle and the hub; and

the rotating guide of the deflection assembly rotates in plane with the first and second axes.

Embodiment 2. The catheter of embodiment 1, further comprising a hub located at the proximal end of the shaft, and wherein the shaft and the hub define an elongate delivery lumen of the catheter, the delivery lumen having a proximal opening, defined by a proximal end of the hub, and a distal opening, formed at a distal end of the shaft and wherein the hub includes a relatively thin wall section extending distally from the proximal opening of the delivery lumen, the thin wall section being located opposite from the handle, and a thickness of the relatively thin wall section being comparable to a wall thickness of the shaft that is in-line therewith.

Embodiment 3. The catheter of any of embodiments 1-2, wherein the hub further includes a slitting guide, the guide comprising a pair of outward protruding ridges between which the relatively thin wall section extends.

Embodiment 4. The catheter of embodiment 1, wherein the proximal end of the hub has a tapered profile that orients the proximal opening of the delivery lumen toward a thumb of a hand whose fingers grasp around the handle.

Embodiment 5. The catheter of embodiment 4, wherein the proximal end of the hub has a relatively soft inner surface being exposed by the tapered profile to form an exposed sealing area.

Embodiment 6. The catheter of any of embodiments 1-5, wherein:

the handle comprises a shell having an outer surface, an inner surface, and a slot extending therethrough from the inner surface to the outer surface, the slot having a length extending along the second longitudinal axis, and the inner surface of the shell including an engagement feature and a support surface formed therein, the engagement feature being located in proximity to the slot, and the support surface being located opposite the slot;

the deflection assembly wire controller comprises a spring biased slider mounted in between the slot and the support surface of the handle shell, the slider including a first side facing toward the support surface, a second side opposite the first side and facing toward the engagement feature of the handle shell, and an operator interface extending from the second side and through the slot to protrude from the outer surface of the handle; and

wherein, when the slider is relaxed according to the spring bias thereof, the second side of the slider interlocks with the engagement feature of the handle shell to prevent movement of the slider along the length of the slot; and

when the slider is deformed against the spring bias thereof, responsive to a force being applied to the operator interface, the second side of the slider moves away from the engagement feature of the handle and no longer interlocks therewith, and the first side of the slider slides along the support surface as the slider moves along the length of the slot, the applied force being along a vector that has a first component generally directed toward the support surface and a second component generally directed along the length of the slot.

Embodiment 7. The catheter of embodiment 6, wherein the inner surface of the handle shell further includes a spindle formed therein, the rotating guide being mounted on the spindle.

Embodiment 8. The catheter of any of embodiments 6-7, wherein: the slider of the deflection assembly wire controller includes a bore formed therein, an opening to the bore being located on the first side of the slider; and the proximal end of the deflection assembly wire extends along the first side of the slider and bends into the bore of the slider for the coupling of the wire controller to the wire.

Embodiment 9. The catheter of claim 8, wherein the proximal end of the deflection assembly wire is reinforced by a sleeve extending thereabout.

Embodiment 10. The catheter of any of claims 6-9, wherein:

the slider of the deflection assembly wire controller comprises first and second parts snap-fit together;

the first part of the slider defines the first and second sides of the slider and includes a bore formed therein, an opening to the bore being located on the first side of the slider;

the proximal end of the deflection assembly wire extends between the first and second parts of the slider and bends into the bore of the first part for the coupling of the wire controller to the wire.

Embodiment 11. The catheter of embodiment 10, wherein the first part of the slider includes opposing cantilever beam members, the beam members defining the spring bias of the slider and a portion of the first side of the slider.

Embodiment 12. The catheter of any of embodiments 6-11, wherein:

the slider of the deflection assembly wire controller comprises a first part, a second part, and an elastomeric core defining the spring bias of the slider, the first and second parts being fitted together, and the core being sandwiched between the first and second parts;

the first part of the slider defines the first side of the slider and includes a bore formed therein, an opening to the bore being located on the first side of the slider;

the second part of the slider defines the second side of the slider; and

the proximal end of the deflection assembly wire extends along the first side of the slider and bends into the bore of the first part for the coupling of the wire controller to the wire.

Embodiment 13. The catheter of embodiment 12, wherein the proximal end of the deflection assembly wire is reinforced by a sleeve extending thereabout.

Embodiment 14. The catheter of embodiment 12, wherein the elastomeric core of the deflection assembly wire controller slider comprises at least one elastic ring member.

Embodiment 15. The catheter of embodiment 14, wherein the at least one elastic ring member comprises first and second elastic ring members stacked together.

Embodiment 16. A catheter comprising a deflectable shaft, a handle, and a deflection assembly, the handle comprising a shell, the handle shell having a length defined from proximal end thereof to a distal end thereof and having an outer surface, an inner surface, and a slot extending therethrough from the inner surface to the outer surface, the distal end of the handle shell being coupled to the shaft proximal end, the slot extending lengthwise along the handle, and the inner surface of the shell including a support surface formed therein, the support surface being located opposite the slot, and the deflection assembly of the catheter comprising an elongate wire extending along the shaft, the wire having a distal end coupled to the shaft in proximity to the distal end thereof and a proximal end extending within the handle shell; and the deflection assembly further comprising:

a wire controller comprising a spring biased slider mounted in between the slot and the support surface of the handle shell, the slider including a first side facing toward the support surface, a second side opposite the first side, an operator interface extending from the second side and through the slot of the handle to protrude from the outer surface of the handle, and an elastomeric core located between the first and second sides and defining the spring bias of the slider; and

wherein the proximal end of the elongate wire is coupled to the slider;

when the slider is relaxed according to the spring bias thereof, the second side of the slider interlocks with the handle shell to prevent movement of the slider along the length of the slot; and

when the slider is deformed against the spring bias thereof, responsive to a force being applied to the operator interface, the second side of the slider moves away from and no longer interlocks with the handle shell, and the first side of the slider slides along the support surface as the slider moves along the length of the slot, the applied force being along a vector that has a first component generally directed toward the support surface and a second component generally directed along the length of the slot, either in a proximal direction or a distal direction.

Embodiment 17. The catheter of embodiment 16, wherein the proximal end of the deflection assembly wire is reinforced by a sleeve extending thereabout.

Embodiment 18. The catheter of any of embodiments 16-17, wherein the slider of the deflection assembly wire controller comprises a first part defining the first side of the slider, a second part defining the second side of the slider, the first and second parts being fitted together, and the elastomeric core being sandwiched between the first and second parts.

Embodiment 19. The catheter of any of embodiments 16-18, wherein the elastomeric core of the deflection assembly wire controller slider comprises at least one elastic ring member.

Embodiment 20. The catheter of any of embodiments 16-19, wherein the at least one elastic ring member comprises first and second elastic ring members stacked together.

Embodiment 21. The catheter of any of embodiment 16-220, wherein the deflection assembly further comprises a rotating guide mounted within the handle shell in proximity to the distal end thereof, the guide including an external groove in which the wire extends between the shaft and the spring biased slider.

Embodiment 22. The catheter of any of embodiments 16-21, wherein:

the deflection assembly wire controller further comprises a spring member;

the wire controller slider further includes a cavity, the cavity having a proximal end oriented toward the proximal end of the handle shell and a distal end oriented toward the distal end of the handle shell, and the spring member being mounted within the cavity, the spring member being biased to push the proximal end of the wire toward the distal end of the cavity, the bias being governed by a predetermined spring force of the spring member; and

when the second component of the applied force vector is in the distal direction, the wire controller slider pushes the wire in a distal direction only if the predetermined spring force of the wire controller spring member is large enough to overcome an opposing force applied along a length of the wire.

Embodiment 23. A deflection assembly for a deflectable shaft catheter, the deflection assembly comprising an elongate wire and a wire controller, the wire controller comprising a spring biased slider, the slider being configured for mounting within a handle of the catheter, the handle comprising a shell, the handle shell having a length defined from a proximal end thereof to a distal end thereof and having an outer surface, an inner surface, and a slot extending therethrough from the inner surface to the outer surface, the distal end of the handle shell being coupled to the shaft, the slot extending lengthwise along the handle, and the inner surface of the shell including an engagement feature and a support surface formed therein, the engagement feature being located in proximity to the slot, and the support surface being located opposite the slot, and the slider comprising:

a first side configured for sliding engagement with the support surface of the handle shell;

a bore having an opening on the first side, the bore being configured to receive a proximal end of the elongate wire and thereby couple the wire to the slider;

a second side opposite the first side and being configured to interlock with the engagement feature of the handle shell;

an operator interface extending from the second side and through the slot of the handle to protrude from the outer surface of the handle; and

an elastomeric core located between the first and second sides and defining the spring bias of the slider.

Embodiment 24. The deflection assembly of embodiment 23, wherein the proximal end of the elongate wire is reinforced by a sleeve extending thereabout.

Embodiment 25. The deflection assembly of embodiment 23, wherein:

the wire controller slider further includes a channel formed in the first side thereof; and

the proximal end of the elongate wire includes a proximal length, a distal length, and a bend extending therebetween, the distal length extending within the channel when the proximal length is received in the bore of the slider.

Embodiment 26. The deflection assembly of any of embodiments 23-25 wherein the wire controller slider further comprises a first part defining the first side of the slider, a second part defining the second side of the slider, the first and second parts being fitted together, and the elastomeric core being sandwiched between the first and second parts.

Embodiment 27. The deflection assembly of any of embodiments 23-26, wherein the elastomeric core of the wire controller slider comprises at least one elastic ring member.

Embodiment 28. The deflection assembly of any of embodiments 23-27, wherein the at least one elastic ring member comprises first and second elastic ring members stacked together.

Embodiment 29. The deflection assembly of any of embodiments 23-28, further comprising a rotating guide configured for mounting within the handle in proximity to the distal end of the handle shell, the guide including an external groove to receive the wire.

Embodiment 30. The deflection assembly of any of embodiment 23-29, wherein:

the wire controller further comprises a spring member;

the wire controller slider further includes a cavity, the cavity having a proximal end and a distal end, and the spring member being mounted within the cavity;

when the slider is mounted within the handle, the cavity of the slider is located between the support surface and engagement feature of the handle shell, the proximal end of the cavity is oriented toward the proximal end of the handle shell, and the distal end of the cavity is oriented toward the distal end of the handle shell; and

the spring member is biased to push the proximal end of the wire toward the distal end of the cavity, the bias being governed by a predetermined spring force of the spring member.

Embodiment 31. A catheter comprising a deflectable shaft, a handle, and a deflection assembly, the handle comprising a shell, the handle shell having a length defined from a proximal end thereof to a distal end thereof and having an outer surface, an inner surface, and a slot extending therethrough from the inner surface to the outer surface, the distal end of the handle shell being coupled to the shaft proximal end, the slot extending lengthwise along the handle, and the inner surface of the shell including a support surface formed therein, the support surface being located opposite the slot, and the deflection assembly of the catheter comprising an elongate wire and a wire controller, the wire extending along the shaft, the wire having a distal end coupled to the shaft in proximity to the distal end thereof and a proximal end extending within the handle, and the wire controller being coupled to the proximal end of the wire within the handle and being mounted between the slot and the support surface of the handle shell, and the wire controller comprising:

a spring biased slider comprising a first side facing toward the support surface of the handle shell, a second side opposite the first side, an operator interface extending from the second side and through the slot to protrude from the outer surface of the handle shell, and a cavity located between the support surface and the engagement feature of the handle shell, the cavity having a proximal end oriented toward the proximal end of the handle shell and a distal end oriented toward the distal end of the handle shell, and the proximal end of the wire being coupled to the slider within the cavity; and

a spring member mounted within the slider cavity and being biased to push the proximal end of the wire toward the distal end of the cavity, the bias being governed by a predetermined spring force of the spring member; and

wherein, when the wire controller slider is relaxed according to the spring bias thereof, the second side of the slider interlocks with the handle shell to prevent movement of the slider along the length of the slot;

when the wire controller slider is deformed against the spring bias thereof, responsive to a force being applied to the operator interface, the second side of the slider moves away from and no longer interlocks with the handle shell and the first side of the slider slides along the support surface as the slider moves along the length of the slot, the applied force being along a vector that has a first component generally directed toward the support surface and a second component generally directed along the length of the slot, either in a proximal direction or a distal direction; and

when the second component of the applied force vector is in the distal direction, the wire controller slider pushes the wire in a distal direction only if the predetermined spring force of the wire controller spring member is large enough to overcome an opposing force applied along a length of the wire.

Embodiment 32. The catheter of embodiment 31, wherein the deflection assembly wire controller further comprises a coupling component mounted within the cavity of the wire controller slider between the wire controller spring member and the distal end of the cavity, the coupling component including a bore into which the proximal end of the deflection assembly wire bends, being secured therein for coupling the wire to the wire controller slider.

Embodiment 33. The catheter of embodiment 32, wherein the proximal end of the deflection assembly wire is reinforced by a sleeve extending thereabout.

Embodiment 34. The catheter of any of embodiments 31-33, wherein the spring biased slider of the deflection assembly wire controller includes opposing cantilever beam members, the beam members defining the spring bias of the slider and a portion of the first side of the slider.

Embodiment 35. The catheter of embodiment 34, wherein each beam member of the slider extends in a direction orthogonal to the length of the handle.

Embodiment 36. The catheter of any of embodiments 31-35, wherein:

the deflection assembly further comprises a rotating guide mounted within the handle between the shaft and the deflection assembly wire controller, the guide including an external groove in which the elongate wire of the deflection assembly extends between the shaft and the wire controller; and

wherein the shaft defines a first longitudinal axis of the catheter and the length of the handle shell defines a second longitudinal axis of the catheter;

the first and second axes cross one another to form an angle between the handle and the shaft; and

the rotating guide of the deflection assembly rotates in plane with the first and second axes.

Embodiment 37. A deflection assembly for a deflectable shaft catheter, the deflection assembly comprising an elongate wire and a wire controller coupled to a proximal end of the wire, the wire controller being configured for mounting within a handle of the catheter, the handle comprising a shell, the handle shell having a length defined from a proximal end thereof to a distal end thereof and having an outer surface, an inner surface, and a slot extending therethrough from the inner surface to the outer surface, the distal end of the handle shell being coupled to the shaft, the slot extending lengthwise along the handle, and the inner surface of the shell including an engagement feature and a support surface formed therein, the engagement feature being located in proximity to the slot, and the support surface being located opposite the slot, and the wire controller comprising:

a spring biased slider comprising a first side, a second side, opposite the first side, an operator interface extending from the second side, and a cavity in which the proximal end of the wire is coupled to the slider, the cavity having a proximal end and a distal end, the slider being sized for mounting within the handle shell so that, when the first side faces toward the support surface of the handle shell, the second side faces toward the engagement feature of the handle shell, the operator interface extends through the slot to protrude from the outer surface of the handle shell, and the cavity is located between the support surface and the engagement feature of the handle shell and the proximal end thereof is oriented toward the proximal end of the handle shell and the distal end thereof is oriented toward the distal end of the handle shell; and

a spring member mounted within the slider cavity and being biased to push the proximal end of the wire toward the distal end of the cavity, the bias being governed by a predetermined spring force of the spring member; and

wherein, when the wire controller is mounted within the handle and the slider thereof is relaxed according to the spring bias thereof, the second side of the slider interlocks with the engagement feature of the handle shell to prevent movement of the slider along the length of the slot;

when the wire controller is mounted within the handle and the slider thereof is deformed against the spring bias thereof, responsive to a force being applied to the operator interface, the second side of the slider moves away from the engagement feature of the handle and no longer interlocks therewith, and the first side of the slider slides along the support surface as the slider moves along the length of the slot, the applied force being along a vector that has a first component generally directed toward the support surface and a second component generally directed along the length of the slot, either in a proximal direction or a distal direction; and

when the second component of the applied force vector is in the distal direction, the mounted slider pushes the wire in a distal direction only if the predetermined spring force of the spring member is great enough to overcome an opposing force applied along a length of the wire.

Embodiment 38. The deflection assembly of embodiment 37, wherein the wire controller further comprises a coupling component mounted within the cavity of the wire controller slider between the wire controller spring member and the distal end of the cavity, the coupling component including a bore into which the proximal end of the deflection assembly wire bends and is secured for coupling the wire to the wire controller slider.

Embodiment 39. The deflection assembly of embodiment 38, wherein the proximal end of the elongate wire is reinforced by a sleeve extending thereabout.

Embodiment 40. The deflection assembly of embodiment 37, wherein the spring biased slider of the wire controller includes opposing cantilever beam members, the beam members defining the spring bias of the slider and a portion of the first side of the slider.

Embodiment 41. The deflection assembly of any of embodiments 37-40, wherein the spring biased slider of the wire controller includes an elastomeric core located between the first and second sides of the slider and defines the spring bias thereof.

In the foregoing detailed description, the invention has been described with reference to specific embodiments. However, it may be appreciated that various modifications and changes can be made without departing from the scope of the invention as set forth in the appended claims. For example, embodiments of deflection assemblies described herein may be incorporated into catheters that do not include elongate delivery lumens like those described herein, and various components and features of each embodiment may be mixed and matched to form additional embodiments.

1. A catheter comprising a deflectable shaft, a handle coupled to shaft and extending laterally therefrom and a deflection assembly, the shaft defining a first longitudinal axis of the catheter, the deflection assembly comprising an elongate wire and a wire controller, the wire extending along the shaft, the wire having a distal end coupled to the shaft in proximity to the distal end thereof and a proximal end extending within the handle, the wire controller being coupled to the proximal end of the wire within the handle; and the deflection assembly further comprising:
a rotating guide mounted within the handle between the shaft and the wire controller, the guide including an external groove in which the wire extends between the shaft and the wire controller; and
wherein the lateral extent of the handle defines a second longitudinal axis of the catheter, the second axis crossing the first axis to form an angle between the handle and the hub; and
the rotating guide of the deflection assembly rotates in plane with the first and second axes.

2. The catheter of claim 1, further comprising a hub located at the proximal end of the shaft, and wherein the shaft and the hub define an elongate delivery lumen of the catheter, the delivery lumen having a proximal opening, defined by a proximal end of the hub, and a distal opening, formed at a distal end of the shaft and wherein the hub includes a relatively thin wall section extending distally from the proximal opening of the delivery lumen, the thin wall section being located opposite from the handle, and a thickness of the relatively thin wall section being comparable to a wall thickness of the shaft that is in-line therewith.

3. The catheter of claim 2, wherein the hub further includes a slitting guide, the guide comprising a pair of outward protruding ridges between which the relatively thin wall section extends.

4. The catheter of claim 1, wherein the proximal end of the hub has a tapered profile that orients the proximal opening of the delivery lumen toward a thumb of a hand whose fingers grasp around the handle.

5. The catheter of claim 4, wherein the proximal end of the hub has a relatively soft inner surface being exposed by the tapered profile to form an exposed sealing area.

6. The catheter of claim 1, wherein:
the handle comprises a shell having an outer surface, an inner surface, and a slot extending therethrough from the inner surface to the outer surface, the slot having a length extending along the second longitudinal axis, and the inner surface of the shell including an engagement feature and a support surface formed therein, the engagement feature being located in proximity to the slot, and the support surface being located opposite the slot;
the deflection assembly wire controller comprises a spring biased slider mounted in between the slot and the support surface of the handle shell, the slider including a first side facing toward the support surface, a second side opposite the first side and facing toward the engagement feature of the handle shell, and an operator interface extending from the second side and through the slot to protrude from the outer surface of the handle; and
wherein, when the slider is relaxed according to the spring bias thereof, the second side of the slider interlocks with the engagement feature of the handle shell to prevent movement of the slider along the length of the slot; and
when the slider is deformed against the spring bias thereof, responsive to a force being applied to the operator interface, the second side of the slider moves away from the engagement feature of the handle and no longer interlocks therewith, and the first side of the slider slides along the support surface as the slider moves along the length of the slot, the applied force being along a vector that has a first component generally directed toward the support surface and a second component generally directed along the length of the slot.

7. The catheter of claim 6, wherein the inner surface of the handle shell further includes a spindle formed therein, the rotating guide being mounted on the spindle.

8. The catheter of claim 6, wherein:
the slider of the deflection assembly wire controller includes a bore formed therein, an opening to the bore being located on the first side of the slider; and
the proximal end of the deflection assembly wire extends along the first side of the slider and bends into the bore of the slider for the coupling of the wire controller to the wire.

9. The catheter of claim 8, wherein the proximal end of the deflection assembly wire is reinforced by a sleeve extending thereabout.

10. The catheter of claim 6, wherein:
the slider of the deflection assembly wire controller comprises first and second parts snap-fit together;
the first part of the slider defines the first and second sides of the slider and includes a bore formed therein, an opening to the bore being located on the first side of the slider;
the proximal end of the deflection assembly wire extends between the first and second parts of the slider and bends into the bore of the first part for the coupling of the wire controller to the wire.

11. The catheter of claim 10, wherein the first part of the slider includes opposing cantilever beam members, the beam members defining the spring bias of the slider and a portion of the first side of the slider.

12. The catheter of claim 6, wherein:
the slider of the deflection assembly wire controller comprises a first part, a second part, and an elastomeric core defining the spring bias of the slider, the first and second parts being fitted together, and the core being sandwiched between the first and second parts;
the first part of the slider defines the first side of the slider and includes a bore formed therein, an opening to the bore being located on the first side of the slider;
the second part of the slider defines the second side of the slider; and
the proximal end of the deflection assembly wire extends along the first side of the slider and bends into the bore of the first part for the coupling of the wire controller to the wire.

13. The catheter of claim 12, wherein the proximal end of the deflection assembly wire is reinforced by a sleeve extending thereabout.

14. The catheter of claim 12, wherein the elastomeric core of the deflection assembly wire controller slider comprises at least one elastic ring member.

15. The catheter of claim 14, wherein the at least one elastic ring member comprises first and second elastic ring members stacked together.

16. A catheter comprising a deflectable shaft, a handle, and a deflection assembly, the handle comprising a shell, the handle shell having a length defined from proximal end thereof to a distal end thereof and having an outer surface, an inner surface, and a slot extending therethrough from the inner surface to the outer surface, the distal end of the handle shell being coupled to the shaft proximal end, the slot extending lengthwise along the handle, and the inner surface of the shell including a support surface formed therein, the support surface being located opposite the slot, and the deflection assembly of the catheter comprising an elongate wire extending along the shaft, the wire having a distal end coupled to the shaft in proximity to the distal end thereof and a proximal end extending within the handle shell; and the deflection assembly further comprising:
a wire controller comprising a spring biased slider mounted in between the slot and the support surface of the handle shell, the slider including a first side facing toward the support surface, a second side opposite the first side, an operator interface extending from the second side and through the slot of the handle to protrude from the outer surface of the handle, and an elastomeric core located between the first and second sides and defining the spring bias of the slider; and
wherein the proximal end of the elongate wire is coupled to the slider;
when the slider is relaxed according to the spring bias thereof, the second side of the slider interlocks with the handle shell to prevent movement of the slider along the length of the slot; and
when the slider is deformed against the spring bias thereof, responsive to a force being applied to the operator interface, the second side of the slider moves away from and no longer interlocks with the handle shell, and the first side of the slider slides along the support surface as the slider moves along the length of the slot, the applied force being along a vector that has a first component generally directed toward the support surface and a second component generally directed along the length of the slot, either in a proximal direction or a distal direction.

17. The catheter of claim 16, wherein the proximal end of the deflection assembly wire is reinforced by a sleeve extending thereabout.

18. The catheter of claim 16, wherein the slider of the deflection assembly wire controller comprises a first part defining the first side of the slider, a second part defining the second side of the slider, the first and second parts being fitted together, and the elastomeric core being sandwiched between the first and second parts.

19. The catheter of claim 16, wherein the elastomeric core of the deflection assembly wire controller slider comprises at least one elastic ring member.

20. The catheter of claim 16, wherein the at least one elastic ring member comprises first and second elastic ring members stacked together.

21. The catheter of claim 16, wherein the deflection assembly further comprises a rotating guide mounted within the handle shell in proximity to the distal end thereof, the guide including an external groove in which the wire extends between the shaft and the spring biased slider.

22. The catheter of claim 16, wherein:
the deflection assembly wire controller further comprises a spring member;
the wire controller slider further includes a cavity, the cavity having a proximal end oriented toward the proximal end of the handle shell and a distal end oriented toward the distal end of the handle shell, and the spring member being mounted within the cavity, the spring member being biased to push the proximal end of the wire toward the distal end of the cavity, the bias being governed by a predetermined spring force of the spring member; and
when the second component of the applied force vector is in the distal direction, the wire controller slider pushes the wire in a distal direction only if the predetermined spring force of the wire controller spring member is large enough to overcome an opposing force applied along a length of the wire.

23. A deflection assembly for a deflectable shaft catheter, the deflection assembly comprising an elongate wire and a wire controller, the wire controller comprising a spring biased slider, the slider being configured for mounting within a handle of the catheter, the handle comprising a shell, the handle shell having a length defined from a proximal end thereof to a distal end thereof and having an outer surface, an inner surface, and a slot extending therethrough from the inner surface to the outer surface, the distal end of the handle shell being coupled to the shaft, the slot extending lengthwise along the handle, and the inner surface of the shell including an engagement feature and a support surface formed therein, the engagement feature being located in proximity to the slot, and the support surface being located opposite the slot, and the slider comprising:
a first side configured for sliding engagement with the support surface of the handle shell;
a bore having an opening on the first side, the bore being configured to receive a proximal end of the elongate wire and thereby couple the wire to the slider;
a second side opposite the first side and being configured to interlock with the engagement feature of the handle shell;
an operator interface extending from the second side and through the slot of the handle to protrude from the outer surface of the handle; and
an elastomeric core located between the first and second sides and defining the spring bias of the slider.

24. The deflection assembly of claim 23, wherein the proximal end of the elongate wire is reinforced by a sleeve extending thereabout.

25. The deflection assembly of claim 23, wherein:
The wire controller slider further includes a channel formed in the first side thereof; and
the proximal end of the elongate wire includes a proximal length, a distal length, and a bend extending therebetween, the distal length extending within the channel when the proximal length is received in the bore of the slider.

26. The deflection assembly of claim 23 wherein the wire controller slider further comprises a first part defining the first side of the slider, a second part defining the second side of the slider, the first and second parts being fitted together, and the elastomeric core being sandwiched between the first and second parts.

27. The deflection assembly of claim 23, wherein the elastomeric core of the wire controller slider comprises at least one elastic ring member.

28. The deflection assembly of claim 23, wherein the at least one elastic ring member comprises first and second elastic ring members stacked together.

29. The deflection assembly of claim 23, further comprising a rotating guide configured for mounting within the handle in proximity to the distal end of the handle shell, the guide including an external groove to receive the wire.

30. The deflection assembly of claim 23, wherein:
the wire controller further comprises a spring member;
the wire controller slider further includes a cavity, the cavity having a proximal end and a distal end, and the spring member being mounted within the cavity;
when the slider is mounted within the handle, the cavity of the slider is located between the support surface and engagement feature of the handle shell, the proximal end of the cavity is oriented toward the proximal end of the handle shell, and the distal end of the cavity is oriented toward the distal end of the handle shell; and
the spring member is biased to push the proximal end of the wire toward the distal end of the cavity, the bias being governed by a predetermined spring force of the spring member.

31. A catheter comprising a deflectable shaft, a handle, and a deflection assembly, the handle comprising a shell, the handle shell having a length defined from a proximal end thereof to a distal end thereof and having an outer surface, an inner surface, and a slot extending therethrough from the inner surface to the outer surface, the distal end of the handle shell being coupled to the shaft proximal end, the slot extending lengthwise along the handle, and the inner surface of the shell including a support surface formed therein, the support surface being located opposite the slot, and the deflection assembly of the catheter comprising an elongate wire and a wire controller, the wire extending along the shaft, the wire having a distal end coupled to the shaft in proximity to the distal end thereof and a proximal end extending within the handle, and the wire controller being coupled to the proximal end of the wire within the handle and being mounted between the slot and the support surface of the handle shell, and the wire controller comprising:
a spring biased slider comprising a first side facing toward the support surface of the handle shell, a second side opposite the first side, an operator interface extending from the second side and through the slot to protrude from the outer surface of the handle shell, and a cavity located between the support surface and the engagement feature of the handle shell, the cavity having a proximal end oriented toward the proximal end of the handle shell and a distal end oriented toward the distal end of the handle shell, and the proximal end of the wire being coupled to the slider within the cavity; and
a spring member mounted within the slider cavity and being biased to push the proximal end of the wire toward the distal end of the cavity, the bias being governed by a predetermined spring force of the spring member; and
wherein, when the wire controller slider is relaxed according to the spring bias thereof, the second side of the slider interlocks with the handle shell to prevent movement of the slider along the length of the slot;
when the wire controller slider is deformed against the spring bias thereof, responsive to a force being applied to the operator interface, the second side of the slider moves away from and no longer interlocks with the handle shell and the first side of the slider slides along the support surface as the slider moves along the length of the slot, the applied force being along a vector that has a first component generally directed toward the support surface and a second component generally directed along the length of the slot, either in a proximal direction or a distal direction; and
when the second component of the applied force vector is in the distal direction, the wire controller slider pushes the wire in a distal direction only if the predetermined spring force of the wire controller spring member is large enough to overcome an opposing force applied along a length of the wire.

32. The catheter of claim 31, wherein the deflection assembly wire controller further comprises a coupling component mounted within the cavity of the wire controller slider between the wire controller spring member and the distal end of the cavity, the coupling component including a bore into which the proximal end of the deflection assembly wire bends, being secured therein for coupling the wire to the wire controller slider.

33. The catheter of claim 32, wherein the proximal end of the deflection assembly wire is reinforced by a sleeve extending thereabout.

34. The catheter of claim 31, wherein the spring biased slider of the deflection assembly wire controller includes opposing cantilever beam members, the beam members defining the spring bias of the slider and a portion of the first side of the slider.

35. The catheter of claim 34, wherein each beam member of the slider extends in a direction orthogonal to the length of the handle.

36. The catheter of claim 31, wherein:
the deflection assembly further comprises a rotating guide mounted within the handle between the shaft and the deflection assembly wire controller, the guide including an external groove in which the elongate wire of the deflection assembly extends between the shaft and the wire controller; and
wherein the shaft defines a first longitudinal axis of the catheter and the length of the handle shell defines a second longitudinal axis of the catheter;
the first and second axes cross one another to form an angle between the handle and the shaft; and
the rotating guide of the deflection assembly rotates in plane with the first and second axes.

37. A deflection assembly for a deflectable shaft catheter, the deflection assembly comprising an elongate wire and a wire controller coupled to a proximal end of the wire, the wire controller being configured for mounting within a handle of the catheter, the handle comprising a shell, the handle shell having a length defined from a proximal end thereof to a distal end thereof and having an outer surface, an inner surface, and a slot extending therethrough from the inner surface to the outer surface, the distal end of the handle shell being coupled to the shaft, the slot extending lengthwise along the handle, and the inner surface of the shell including an engagement feature and a support surface formed therein, the engagement feature being located in proximity to the slot, and the support surface being located opposite the slot, and the wire controller comprising:
a spring biased slider comprising a first side, a second side, opposite the first side, an operator interface extending from the second side, and a cavity in which the proximal end of the wire is coupled to the slider, the cavity having a proximal end and a distal end, the slider being sized for mounting within the handle shell so that, when the first side faces toward the support surface of the handle shell, the second side faces toward the engagement feature of the handle shell, the operator interface extends through the slot to protrude from the outer surface of the handle shell, and the cavity is located between the support surface and the engagement feature of the handle shell and the proximal end thereof is oriented toward the proximal end of the handle shell and the distal end thereof is oriented toward the distal end of the handle shell; and
a spring member mounted within the slider cavity and being biased to push the proximal end of the wire toward the distal end of the cavity, the bias being governed by a predetermined spring force of the spring member; and
wherein, when the wire controller is mounted within the handle and the slider thereof is relaxed according to the spring bias thereof, the second side of the slider interlocks with the engagement feature of the handle shell to prevent movement of the slider along the length of the slot;
when the wire controller is mounted within the handle and the slider thereof is deformed against the spring bias thereof, responsive to a force being applied to the operator interface, the second side of the slider moves away from the engagement feature of the handle and no longer interlocks therewith, and the first side of the slider slides along the support surface as the slider moves along the length of the slot, the applied force being along a vector that has a first component generally directed toward the support surface and a second component generally directed along the length of the slot, either in a proximal direction or a distal direction; and
when the second component of the applied force vector is in the distal direction, the mounted slider pushes the wire in a distal direction only if the predetermined spring force of the spring member is great enough to overcome an opposing force applied along a length of the wire.

38. The deflection assembly of claim 37, wherein the wire controller further comprises a coupling component mounted within the cavity of the wire controller slider between the wire controller spring member and the distal end of the cavity, the coupling component including a bore into which the proximal end of the deflection assembly wire bends and is secured for coupling the wire to the wire controller slider.

39. The deflection assembly of claim 38, wherein the proximal end of the elongate wire is reinforced by a sleeve extending thereabout.

40. The deflection assembly of claim 37, wherein the spring biased slider of the wire controller includes opposing cantilever beam members, the beam members defining the spring bias of the slider and a portion of the first side of the slider.

41. The deflection assembly of claim 37, wherein the spring biased slider of the wire controller includes an elastomeric core located between the first and second sides of the slider and defines the spring bias thereof.
